(19) United States
(12) Patent Application Publication
(10) Pub. No.: US 2010/0151542 A1
(43) Pub. Date: Jun. 17, 2010

(54) Cleaning Enzymes and Fragrance Production

(76) Inventors: Joseph C. McAuliffe, Sunnyvale, CA (US); Jorn Dalgaard Mikkelsen, Copenhagen (DK); Ayrookaran J. Poulose, Belmont, CA (US); Jorn Borch Soe, Tilst (DK)

Correspondence Address: DANISCO US INC.;ATTENTION: LEGAL DEPARTMENT, 925 PAGE MILL ROAD, PALO ALTO, CA, 94304, US

(21) Appl. No.: 12/528,968

(22) PCT Filed: Feb. 27, 2008
(86) PCT No.: PCT/US08/02681

§ 371 Date: Feb. 4, 2010

Related U.S. Application Data
(60) Provisional application No. 60/903,980, filed on Feb. 27, 2007.

(51) Int. Cl. C12P 7/62; C12N 9/10

(52) U.S. Cl. 435/135; 435/193

(57) ABSTRACT

The present invention provides compositions comprising an acyltransferase and an alcohol substrate for the acyl-transferase. In some particularly preferred embodiments, the composition finds use in production of a fragrant ester. In some other embodiments, the composition finds use in laundry detergents to clean stains that contain at least one triglyceride. In some further embodiments, the compositions are used to produce compounds with cleaning properties (e.g., a surfactant ester).

### FIELD OF THE INVENTION

The present invention provides compositions comprising an acyltransferase and an alcohol substrate for the acyltransferase. In some particularly preferred embodiments, the composition finds use in production of a fragrant ester. In some other embodiments, the composition finds use in laundry detergents to clean stains that contain at least one triglyceride. In some further embodiments, the compositions are used to produce compounds with cleaning properties (e.g., a surfactant ester).

### BACKGROUND OF THE INVENTION

When clothes, particularly clothes that are stained with a dairy product (e.g., milk, ice-cream or butter), are washed in a laundry detergent that contains lipase, an unpleasant smell that resembles the odor of baby “spit-up” or rancid butter often emanates from the fabric after the clothes have been dried. It is believed that the malodor is produced by lipase-catalyzed hydrolysis of short chain triglycerides (e.g., C4 to C12-containing triglycerides) that are present in the fabric and/or wash. This hydrolysis reaction produces unpleasant smelling, short chain fatty acids (e.g., butyric acid) which are volatile and cause a persistent malodor. Despite much research in the prevention of malodor and/or imparting pleasant fragrance to laundry, there still remains a need in the art for laundry compositions that address this issue.

### SUMMARY OF THE INVENTION

The present invention provides compositions comprising an acyltransferase and an alcohol substrate for the acyltransferase. In some particularly preferred embodiments, the composition finds use in production of a fragrant ester. In some other embodiments, the composition finds use in laundry detergents to clean stains that contain at least one triglyceride. In some further embodiments, the compositions are used to produce compounds with cleaning properties (e.g., a surfactant ester).

The present invention provides compositions for producing a fragrant ester, comprising the following components: an SGNH acyltransferase, an alcohol substrate for the SGNH acyltransferase, and an acyl donor, wherein the SGNH acyltransferase catalyzes transfer of an acyl group from the acyl donor to the alcohol substrate to produce a fragrant ester in an aqueous environment. In some embodiments, the composition is an aqueous composition that further comprises a fragrant ester. In some further embodiments, the alcohol substrate and acyl donor and chosen to produce a particular fragrant ester. In some preferred embodiments, the acyl donor is a C1 to C10 acyl donor. In some alternative embodiments, the SGNH acyltransferase is immobilized on a solid support. In some further embodiments, the composition is a dehydrated composition, and wherein the fragrant ester is produced upon rehydration of the composition. In some still additional embodiments, the composition is a foodstuff. In yet further embodiments, the composition is a cleaning composition comprising at least one surfactant. In some additional embodiments, the compositions are cleaning compositions comprising hydrogen peroxide.

The present invention also provides methods for producing at least one fragrant ester, comprising combining: an SGNH acyltransferase, an alcohol substrate for the SGNH acyltransferase, and an acyl donor, wherein the SGNH acyltransferase catalyzes transfer of an acyl group from the acyl donor onto the alcohol substrate to produce at least one fragrant ester. In some embodiments, the alcohol substrate and acyl donor are selected to produce a particular fragrant ester. In some further embodiments, the acyl donor is a C2 to C10 acyl donor. In yet additional embodiments, the methods comprise rehydrating the components after they are combined. In some alternative embodiments, the rehydration occurs during mastication. In still further embodiments, the SGNH acyltransferase, alcohol substrate, and acyl donor are combined in an aqueous environment. In some alternative embodiments, the SGNH acyltransferase is immobilized on a solid support.

The present invention also provides methods for the simultaneous generation of a bleaching agent and a fragrance comprising combining: an SGNH acyltransferase, an alcohol substrate for the SGNH acyltransferase, and an acyl donor, wherein the SGNH acyltransferase catalyzes transfer of an acyl group from the acyl donor onto the alcohol substrate to produce fragrance and a bleaching agent. In some embodiments, the bleaching agent is a peracid. In some particularly preferred embodiments, the peracid is peracetic acid. In some further preferred embodiments, the SGNH acyltransferase is M. smegmatis acyltransferase. In some additional preferred embodiments, the fragrance is an ester. In some still further embodiments, the ester is a C2 to C6 ester of a primary alcohol. In yet additional embodiments, application of the bleaching agent to a stain results in removal of the stain.

The present invention provides cleaning compositions that comprise an acyltransferase (e.g., an SGNH acyltransferase) and an alcohol substrate for the acyltransferase.

In some of these embodiments, the acyltransferase and alcohol substrate are present in amounts effective to produce a detectable ester upon contact of the cleaning composition with an acyl donor-containing object. In some embodiments, the cleaning composition further comprises an acyl donor-containing object and an ester that is produced as result of a reaction, catalyzed by the acyltransferase, between the alcohol substrate and the acyl donor. In some preferred embodiments, the acyl donor is a C1 to C10 acyl donor.

In some other embodiments, the cleaning composition also comprises an added acyl donor (e.g., triglyceride, fatty acid ester or the like) which reacts with the alcohol substrate. In some particularly preferred embodiments, the ester produced by the composition is a fragrant ester, a surfactant ester, a surfactant, or fabric care agent, or combinations of these.

In some embodiments, the acyl donor-containing object is soiled with the acyl donor. In some preferred embodiments, the acyl donor is an oily substance, such as an animal fat, plant fat, dairy product or the like. In some further preferred embodiments, the combination of the acyl donor and the alcohol substrate results in the production of a fragrant ester, a surfactant ester, a water soluble ester, or a fabric care agent, or any combination thereof. Indeed, it intended that the present invention provide a combination of benefits.

In some embodiments, the cleaning composition further comprises at least one lipase. In some additional embodiments, the cleaning composition further comprises at least one surfactant and/or at least one source of peroxide. In some embodiments, the surfactant or emulsifying agent of the cleaning composition acts on the alcohol substrate for acyl transfer.

In some further embodiments, the cleaning compositions of the present invention further comprise at least one additional enzyme, including but not limited to hemicellulases, peroxidases, proteases, cellulases, xylanases, lipases, phospholipases, esterases, cutinases, pectinases, pectate lyases, amylases, mannanases, keratinases, reductases, oxidases, phenoloxidases, lipoxygenases, ligninases, pullulanases, tannases, pentosanases, malanases, beta-glucanases, arabinosidases, hyaluronidase, chondroitinase, laccase, and amylases, or mixtures thereof. In some embodiments, a combination of enzymes (i.e., a “cocktail”) comprising conventional applicable enzymes like protease, lipase, cutinase and/or cellulase in conjunction with acyltransferase is used.

In some further embodiments, the cleaning compositions further comprise at least one surfactant, builder, polymer, salt, bleach activator, solvent, buffer, or perfume etc, as described in greater detail herein.

In some embodiments, the cleaning composition is an aqueous composition. In some preferred embodiments, the cleaning composition comprises at least about 90% water, excluding any solid components.

The present invention also provides cleaning methods that utilize the cleaning compositions provided herein. These methods generally comprise combining an acyltransferase (e.g., an SGNH acyltransferase, an alcohol substrate for the acyltransferase, and an object (e.g., a fabric) soiled with an acyl donor-containing substance, wherein the acyltransferase catalyzes transfer of an acyl group from the acyl donor onto the alcohol substrate to produce an ester.

In some embodiments, the object is soiled with an oil-containing substance (e.g., a triacylglyceride-containing substance, such as a substance that contains C4-C18 triacylglycerides). In some preferred embodiments, the combination of the oil-containing substance and the alcohol, the ester produced is a fragrant ester, while in other embodiments, a non-fragrant ester is produced, and in still other embodiments, a surfactant or other fabric care agent, or combinations of these esters are produced.

In some of these embodiments, use of the acyltransferase enzyme reduces the amount of malodor that is typically produced by hydrolysis of triglycerides, by synergistically working with a lipase enzyme to increase the rate of removal of acyl chains from triacylglyceride; and/or linking the acyl chains to an alcohol substrate, thus forming an ester product rather than a volatile fatty acid.

In some particularly preferred embodiments, the present invention also provides compositions for producing fragrant esters. In some embodiments, the compositions comprise an acyltransferase (e.g., an SGNH acyltransferase), an alcohol substrate for the acyltransferase, and an acyl donor, wherein the acyltransferase catalyzes transfer of an acyl group from the acyl donor to the alcohol substrate to produce a fragrant ester in an aqueous environment. In some particularly preferred embodiments, the alcohol substrate and the acyl donor are utilized to produce a particular fragrant ester. In some embodiments, the composition is an aqueous composition that further comprises the fragrant ester. In some other embodiments, the composition is a dehydrated composition, wherein the fragrant ester is produced upon subsequent rehydration of the composition.

In some embodiments, the acyl donor donates a C1 to C10 acyl chain to the alcohol substrate. In some particularly preferred embodiments, the compositions for producing fragrant esters are cleaning compositions.

In some embodiments, the acyltransferase is immobilized on a solid support.

In some further embodiments, the composition comprises a foodstuff. In some other embodiments, the composition is a cleaning composition. In some yet additional embodiments, the composition further contains at least one surfactant.

The present invention also provides methods that utilize the compositions provided herein to produce at least one fragrant ester. In general, these methods comprise combining an acyltransferase (e.g., an SGNH acyltransferase), an alcohol substrate for the acyltransferase, and an acyl donor, wherein the acyltransferase catalyzes transfer of an acyl group from the acyl donor onto the alcohol substrate to produce the fragrant ester. In some embodiments, the alcohol substrate and the acyl donor produce a particular fragrant ester.

In some embodiments in which the compositions are dehydrated, the methods further comprise the step of rehydrating the components after they are combined. In some embodiments, rehydration occurs by the addition of any suitable aqueous medium, including water, milk or saliva. Thus, in some embodiments, rehydration occurs during mastication, to release a fragrant ester. In some other embodiments, the alcohol substrate and the acyl donor are combined in an aqueous environment.

### BRIEF DESCRIPTION OF THE FIGURES

Certain aspects of the following detailed description are best understood when read in conjunction with the accompanying drawings. It is emphasized that, according to common practice, the various features of the drawings are not to-scale. On the contrary, the dimensions of the various features are arbitrarily expanded or reduced for clarity.

FIG. 1 provides graphs showing the conversion of cis-3-hexenol, 2-phenylethanol and 2-methyl-1-butanol to their respective butyryl esters with tributyrin and two acyltransferases.

FIG. 2 provides graphs showing a comparison of free and sol-gel encapsulated forms of acyltransferase (AcT) for the esterification of cis-3-hexenol with triacetin at 10, 30 and 120 minutes.

FIG. 3 provides a panel of graphs of LC/MS data showing transesterification of tetraethyleneglycol using tributyrin and AcT in a detergent background.

FIG. 4 provides a panel of graphs of LC/MS data showing transesterification of ¹³C-U-glycerol using tributyrin and AcT in a detergent background.

FIG. 5 provides a graph showing production of benzyl butyrate from butterfat and benzyl alcohol in the presence of lipases and AcT.

FIG. 6 provides an illustration of an exemplary method for producing fragrant esters from butterfat.

FIG. 7 provides results of TLC analysis of lipid from incubation of egg yolk/sorbitol with 1) KLM3 mutant pLA231 and 2) control. In this Figure, “PE” is phosphatidylethanolamine, and “PC” is phosphatidylcholine.

FIG. 8 provides a GLC chromatogram of sample 2467-112-1, egg yolk/sorbitol treated with KLM3, pLA231.

FIG. 9 provides a GLC chromatogram of sample 2467-112-2, egg yolk/sorbitol control sample.

FIG. 10 provides a GLC/MS spectrum of sorbitol monooleate identified from Grindsted SMO and MS spectrum of the identified peak in egg yolk/sorbitol treated with KLM3 pLA 231(2467-112-1).

### DESCRIPTION OF THE INVENTION

The present invention provides compositions comprising an acyltransferase and an alcohol substrate for the acyltransferase. In some particularly preferred embodiments, the composition finds use in production of a fragrant ester. In some other embodiments, the composition finds use in laundry detergents to clean stains that contain at least one triglyceride. In some further embodiments, the compositions are used to produce compounds with cleaning properties (e.g., a surfactant ester).

Unless otherwise indicated, the practice of certain aspects of the present invention involves conventional techniques commonly used in molecular biology, microbiology, protein purification, protein engineering, protein and DNA sequencing, and recombinant DNA fields, which are within the skill of the art. All patents, patent applications, articles and publications mentioned herein, both supra and infra, are hereby expressly incorporated herein by reference.

Furthermore, the headings provided herein are not limitations of the various aspects or embodiments of the invention which can be had by reference to the specification as a whole. Accordingly, the terms set forth immediately below are more fully defined by reference to the specification as a whole. Nonetheless, in order to facilitate understanding of the invention, a number of terms are defined below.

### DEFINITIONS

Unless defined otherwise herein, all technical and scientific terms used herein have the same meaning as commonly understood by one of ordinary skill in the art to which this invention pertains. Although any methods and materials similar or equivalent to those described herein find use in the practice of what is described herein, exemplary methods and materials are described herein. As used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the term “acyl group” refers to an organic group of the formula (RC═O—).

As used herein, the term “acylation” refers to the chemical reaction that transfers the acyl (RCO—) group from one molecule (an “acyl donor”) onto another molecule (a “substrate”), generally, by substituting a hydrogen of an —OH group of the substrate with the acyl group.

As used herein, the term “acyl donor” refers to the molecule that donates an acyl group in an acyltransferase reaction.

As used herein, the term “alcohol substrate” refers to any organic molecule comprising a reactive hydroxyl group (—OH) bound to a carbon atom. This term excludes polysaccharides and proteins. Water is not an alcohol substrate. Exemplary alcohol substrates include, but are not limited to aliphatic alcohols, alicyclic or aromatic alcohols, terpene alcohols, and polyols including monomeric, dimeric, trimeric and tetrameric polyols. In some embodiments, an alcohol contains more than one hydroxyl group. Alcohol substrates are capable of receiving an acyl group in the acyltransferase reaction described below. In some embodiments, the alcohol is a primary, secondary or tertiary alcohol.

As used herein, the term “transferase” refers to an enzyme that catalyzes the transfer of functional compounds to a range of substrates.

The term “acyltransferase” as used herein refers to any enzyme generally classified as E.C. 2.3.1.x that is capable of transferring an acyl group from an acyl donor, (e.g., a lipid), onto an alcohol substrate.

As used herein, the term “GDSX acyltransferase” refers to an acyltransferase having a distinct active site that contains a GDSX sequence motif (in which X is often L), usually near the N-terminus. GDSX enzymes have five consensus sequences (I-V). These enzymes are known (See e.g., Upton et al., Trends Biochem. Sci., 20:178-179 [1995]; and Akoh et al., Prog. Lipid Res., 43:534-52 [2004]). A sub-set of GDSX acyltransferases contain conserved SG and H residues in the consensus sequences. These GDSX acyltransferases are “SGNH acyltransferases.”

As used herein, the term “SGNH acyltransferase” refers to an acyltransferase of the SGNH hydrolase family, wherein members of the SGNH hydrolase family contain a SGNH hydrolase-type esterase domain, which has a three-layer alpha/beta/alpha structure, where the beta-sheets are composed of five parallel strands. Enzymes containing this domain act as esterases, lipases and acyltransferases, but have little sequence homology to classical lipases (See, Akoh et al., Prog. Lipid Res., 43:534-552 [2004]; and Wei et al., Nat. Struct. Biol., 2: 218-223 [1995]).

Proteins containing an SGNH hydrolase-type esterase domain have been found in a variety of species and include, but are not limited to an esterase from Streptomyces scabies (See, Sheffield et al., Protein Eng., 14:513-519 [2001]); the esterase of viral haemagglutinin-esterase surface glycoproteins from influenza C virus, coronaviruses and toroviruses (See, Molgaard et al., Acta Crystallogr. D 58:111-119 [2002]); mammalian acetylhydrolases (See, Lo et al., J. Mol. Biol., 330:539-551 [2003]); fungal rhamnogalacturonan acetylesterase (See, Molgaard et al., Structure 8:373-383 [2000]); and the multifunctional enzyme thioesterase I (TAP) from Escherichia coli (See, Molgaard et al., Acta Crystallogr. D 60: 472-478 [2004]). SGNH hydrolase-type esterase domains contain a unique hydrogen bond network that stabilizes their catalytic centers. In some preferred embodiments, they contain a conserved Ser/Asp/His catalytic triad. SGNH acyltransferases are also described in accession number cd01839.3 in the conserved domain database of the GENBANK® database (incorporated by reference herein). SGNH acyltransferases form an acyl-enzyme intermediate upon contact with an acyl donor, and transfer the acyl group to an acceptor other than water.

As used herein, the term “classical lipase” refers to an enzyme having lipase activity and a signature GXSXG motif that contains the active site serine (See e.g., Derewenda et al., Biochem Cell Biol., 69:842-51 [1991]). In some embodiments, the classical lipase is a triacylglyceride lipase that has specificity for the sn1 and sn3 positions of a triacylglyceride.

SGNH acyltransferases and GDSL acyltransferases have a similar structure, and both are structurally distinct from classical lipases.

The term “transesterification” as used herein, refers to the enzyme catalyzed transfer of an acyl group from a lipid donor (other than a free fatty acid) to an acyl acceptor (other than water).

As used herein, the term “alcoholysis” refers to the enzyme catalyzed cleavage of a covalent bond of an acid derivative by reaction with an alcohol ROH so that one of the products combines with the H of the alcohol and the other product combines with the OR group of the alcohol.

As used herein, the term “hydrolysis” refers to the enzyme catalyzed transfer of an acyl group from a lipid to the OH group of a water molecule.

As used herein, the term “aqueous,” as used in the phrases “aqueous composition” and “aqueous environment” refers to a composition that is made up of at least about 50% water. In some embodiments, aqueous compositions comprise at least about 50% water, at least about 60% water, at least about 70% water, at least about 80% water, at least about 90% water, at least about 95% water, or at least about 97% water. In some embodiments, a portion of the remainder of an aqueous composition comprises at least one alcohol.

In some preferred embodiments, the term “aqueous,” refers to a composition having a water activity (A_{w}) of at least about 0.75, at least about 0.8, at least about 0.9, or at least about 0.95, as compared to distilled water.

As used herein, the term “fragrant ester” refers to an ester that has a pleasant aroma or taste. This term encompasses both fragrant esters and flavorsome esters. Such esters are well known in the art.

As used herein, the term “fabric care agent” refers to a compound that has a cleaning property and/or imparts a benefit to fabric. Such compounds include surfactants and emulsifiers. In some embodiments, the fabric care agents impart benefits such as softening, improvement in the fabric feel, de-pilling, color retention, etc.

As used herein, the term “surfactant ester” refers to an ester that has surfactant properties, wherein a surfactant is a compound that lowers the surface tension of a liquid.

As used herein, the term “detectably fragrant” refers to an amount of a fragrant ester that is detectable by a human nose or taste buds. A fragrant ester that is present in an amount that is only detectable by a mass spectrometer, but not by the human nose or taste bud, is not detectably fragrant.

As used herein, the term “object” refers to an item that is to be cleaned. It is intended that the present invention encompass any object suitable for cleaning, including but not limited to fabrics (e.g., clothing), upholstery, carpeting, hard surfaces (e.g., countertops, floors, etc.), or dishware (e.g., plates, cups, saucers, bowls, cutlery, silverware, etc.).

As used herein, the term “stained” or “soiled” refers to an object that is dirty. The stain does not have to be visible to the human eye for the object to be stained. For example, a stained or soiled object refers to an object (e.g., a fabric), containing a fatty substance from an animal (e.g., a dairy product), plant, human sweat, etc.

As used herein, the term “dairy product” refers to milk (e.g., whole, reduced fat, nonfat milk, or buttermilk), or a product made therefrom such as cheese of any type (e.g., cream cheese, hard cheese, soft cheese, etc.), butter, yogurt, and ice-cream. Indeed, it is not intended that the present invention be limited to any specific dairy product, as any milk-based product is encompassed by this definition.

As used herein, the term “acyl donor-containing object” refers to an object that comprises an acyl donor (e.g., a triglyceride). In some embodiments, the acyl donor is present as a stain.

As used herein, the term “immobilized,” in the context of an immobilized enzyme, refers to an enzyme that is affixed (e.g., tethered), to a substrate (e.g., a solid or semi-solid support), and not free in solution.

As used herein, the term “in solution” refers to a molecule (e.g., an enzyme), that is not immobilized on a substrate and is free in a liquid composition.

As used herein, the terms “amounts effective” and “effective amount” in the context of the phrase “an amount effective to produce a detectable ester” refers to an amount of a component (e.g., enzyme, substrate, acyl donor, or any combination thereof), to produce a desired product under the conditions used.

As used herein, the term “source of hydrogen peroxide” includes hydrogen peroxide as well as the components of a system that can spontaneously or enzymatically produce hydrogen peroxide as a reaction product.

As used herein, “personal care products” means products used in the cleaning, bleaching and/or disinfecting of hair, skin, scalp, and teeth, including, but not limited to shampoos, body lotions, shower gels, topical moisturizers, toothpaste, and/or other topical cleansers. In some particular embodiments, these products are utilized on humans, while in other embodiments, these products find use with non-human animals (e.g., in veterinary applications).

As used herein, “cleaning compositions” and “cleaning formulations” refer to compositions that find use in the removal of undesired compounds from items to be cleaned, such as fabric, dishes, contact lenses, other solid substrates, hair (shampoos), skin (soaps and creams), teeth (mouthwashes, toothpastes) etc. The term encompasses any materials/compounds selected for the particular type of cleaning composition desired and the form of the product (e.g., liquid, gel, granule, or spray composition), as long as the composition is compatible with the acyltransferase and any other enzyme(s) and/or components present in the composition. The specific selection of cleaning composition materials are readily made by considering the object/surface to be cleaned, and the desired form of the composition for the cleaning conditions employed during use.

The terms further refer to any composition that is suited for cleaning, bleaching, disinfecting, and/or sterilizing any object and/or surface. It is intended that the terms include, but are not limited to detergent compositions (e.g., liquid and/or solid laundry detergents and fine fabric detergents; hard surface cleaning formulations suitable for use in cleaning glass, wood, ceramic and metal counter tops and windows, etc.; carpet cleaners; oven cleaners; fabric fresheners; fabric softeners; and textile and laundry pre-spotters, as well as dish detergents).

Indeed, the term “cleaning composition,” unless otherwise indicated, as used herein includes, granular or powder-form all-purpose or heavy-duty washing agents, especially cleaning detergents; liquid, gel or paste-form all-purpose washing agents, especially heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, car or carpet shampoos, bathroom cleaners; hair shampoos and hair-rinses; shower gels and foam baths and metal cleaners; as well as cleaning auxiliaries such as bleach additives and “stain-stick,” pre-treatment,” and/or “pre-wash” types.

As used herein, the terms “detergent composition” and “detergent formulation” are used in reference to mixtures which are intended for use in a wash medium for the cleaning of soiled objects. In some embodiments, the term is used in reference to laundering fabrics and/or garments (e.g., “laundry detergents”). In some alternative embodiments, the term refers to other detergents, such as those used to clean dishes, silverware, cutlery, etc. (e.g., “dishwashing detergents”). It is not intended that the present invention be limited to any particular detergent formulation or composition. Indeed, it is intended that in addition to acyltransferase, the term encompasses detergents that contain surfactants, other transferase(s), hydrolytic and other enzymes, oxido reductases, builders, bleaching agents, bleach activators, bluing agents and fluorescent dyes, caking inhibitors, masking agents, enzyme activators, antioxidants, and solubilizers.

As used herein the term “hard surface cleaning composition,” refers to detergent compositions for cleaning hard surfaces, such as floors, countertops, cabinets, walls, tile, bath and kitchen fixtures, and the like. Such compositions are provided in any form, including but not limited to solids, liquids, emulsions, etc.

As used herein, “dishwashing composition” refers to all forms of compositions for cleaning dishes and other utensils intended for use in food consumption and/or food handling, including but not limited to gel, granular and liquid forms.

As used herein, “fabric cleaning composition” refers to all forms of detergent compositions for cleaning fabrics, including but not limited to gel, granular, liquid and bar forms.

As used herein, “textile” refers to woven fabrics, as well as staple fibers and filaments suitable for conversion to or use as yarns, woven, knit, and non-woven fabrics. The term encompasses yarns made from natural, as well as synthetic (e.g., manufactured) fibers.

As used herein, “textile materials” is a general term for fibers, yarn intermediates, yarn, fabrics, and products made from fabrics (e.g., garments and other articles).

As used herein, “fabric” encompasses any textile material. Thus, it is intended that the term encompass garments, as well as fabrics, yarns, fibers, non-woven materials, natural materials, synthetic materials, and any other textile material.

As used herein, the term “compatible,” means that the cleaning composition materials do not reduce the enzymatic activity of the acyltransferase to such an extent that the acyltransferase is not effective as desired during normal use situations. Specific cleaning composition materials are exemplified in detail hereinafter.

As used herein, “effective amount of enzyme” refers to the quantity of enzyme necessary to achieve the enzymatic activity required in the specific application (e.g., personal care product, cleaning composition, etc.). Such effective amounts are readily ascertained those of ordinary skill in the art and are based on many factors, such as the particular enzyme or variant used, the cleaning application, the specific composition of the cleaning composition, and whether a liquid, gel or dry (e.g., granular, bar) composition is required, etc.

As used herein, “non-fabric cleaning compositions” encompass hard surface cleaning compositions, dishwashing compositions, personal care cleaning compositions (e.g., oral cleaning compositions, denture cleaning compositions, personal cleansing compositions, etc.), and compositions suitable for use in the pulp and paper industry.

As used herein, the term “enzymatic conversion” refers to the modification of a substrate to an intermediate or the modification of an intermediate to an end-product by contacting the substrate or intermediate with an enzyme. In some embodiments, contact is made by directly exposing the substrate or intermediate to the appropriate enzyme. In some other embodiments, contacting comprises exposing the substrate or intermediate to an organism that expresses and/or excretes the enzyme, and/or metabolizes the desired substrate and/or intermediate to the desired intermediate and/or end-product, respectively.

As used herein, “protein of interest,” refers to a protein (e.g., an enzyme or “enzyme of interest”) which is being analyzed, identified and/or modified. Naturally-occurring, as well as recombinant proteins of interest find use in the present invention.

As used herein, “protein” refers to any composition comprised of amino acids and recognized as a protein by those of skill in the art. The terms “protein,” “peptide” and polypeptide are used interchangeably herein. Wherein a peptide is a portion of a protein, those skilled in the art understand the use of the term in context.

As used herein, functionally and/or structurally similar proteins are considered to be “related proteins.” In some embodiments, these proteins are derived from a different genus and/or species, including differences between classes of organisms (e.g., a bacterial protein and a fungal protein). In some embodiments, these proteins are derived from a different genus and/or species, including differences between classes of organisms (e.g., a bacterial enzyme and a fungal enzyme). In additional embodiments, related proteins are provided from the same species. Indeed, it is not intended that the present invention be limited to related proteins from any particular source(s). In addition, the term “related proteins” encompasses tertiary structural homologs and primary sequence homologs. In further embodiments, the term encompasses proteins that are immunologically cross-reactive.

As used herein, the term “derivative” refers to a protein which is derived from a protein by addition of one or more amino acids to either or both the C- and N-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence. The preparation of a protein derivative is may be achieved by modifying a DNA sequence which encodes for the native protein, transformation of that DNA sequence into a suitable host, and expression of the modified DNA sequence to form the derivative protein.

Related (and derivative) proteins comprise “variant proteins.” In some embodiments, variant proteins differ from a parent protein and one another by a small number of amino acid residues. The number of differing amino acid residues may be one or more (e.g., about 1, about 2, about 3, about 4, about 5, about 10, about 15, about 20, about 30, about 40, about 50, or more) amino acid residues. In some embodiments, the number of different amino acids between variants is between about 1 and about 10. In some particular embodiments, related proteins and particularly variant proteins comprise at least about 35%, about 40%, about 45%, about 50%, about 55%, about 60%, about 65%, about 70%, about 75%, about 80%, about 85%, about 90%, about 95%, about 97%, about 98%, or about 99% amino acid sequence identity. Additionally, a related protein or a variant protein as used herein refers to a protein that differs from another related protein or a parent protein in the number of prominent regions. For example, in some embodiments, variant proteins have about 1, about 2, about 3, about 4, about 5, or about 10 corresponding prominent regions that differ from the parent protein.

Several methods are known in the art that are suitable for generating variants of the enzymes of the present invention, including but not limited to site-saturation mutagenesis, scanning mutagenesis, insertional mutagenesis, random mutagenesis, site-directed mutagenesis, and directed-evolution, as well as various other recombinatorial approaches.

In some embodiments, homologous proteins are engineered to produce enzymes with the desired activity(ies). In some embodiments, the engineered proteins are included within the SGNH-hydrolase family of proteins. In some embodiments, the engineered proteins comprise at least one or a combination of the following conserved residues: L6, W14, W34, L38, R56, D62, L74, L78, H81, P83, M90, K97, G110, L114, L135, F180, G205. In alternative embodiments, these engineered proteins comprise the GDSL-GRTT and/or ARTT motifs. In further embodiments, the enzymes are multimers, including but not limited to dimers, octamers, and tetramers.

In some embodiments, in order to establish homology to a primary structure, the amino acid sequence of an acyltransferase is directly compared to the primary amino acid sequence of an acyltransferase and to a set of residues known to be invariant in all acyltransferases for which the sequence is known. After aligning the conserved residues, allowing for necessary insertions and deletions in order to maintain alignment (i.e., avoiding the elimination of conserved residues through arbitrary deletion and insertion), the residues equivalent to particular amino acids in the primary sequence of an acyltransferase are defined. In some embodiments, alignment of conserved residues define 100% of the equivalent residues. However, alignment of greater than about 75% or as little as about 50% of conserved residues are also adequate to define equivalent residues. In some embodiments, conservation of the catalytic serine and histidine residues are maintained.

In some embodiments, conserved residues find use in defining the corresponding equivalent amino acid residues of M. smegmatis acyltransferase in other acyltransferases (e.g., acyltransferases from other Mycobacterium species, as well as any other organisms).

In some embodiments of the present invention, the DNA sequence encoding M. smegmatis acyltransferase provided in WO 05/056782 is modified. In some embodiments, the following residues are modified: Cys7, Asp10, Ser11, Leu12, Thr13, Trp14, Trp16, Pro24, Thr25, Leu53, Ser54, Ala55, Thr64, Asp65, Arg67, Cys77, Thr91, Asn94, Asp95, Tyr99, Val125, Pro138, Leu140, Pro146, Pro148, Trp149, Phe150, Ile153, Phe154, Thr159, Thr186, Ile192, Ile194, and Phe196. However, it is not intended that the present invention be limited to sequence that are modified at these positions. Indeed, it is intended that the present invention encompass various modifications and combinations of modifications.

In some additional embodiments, equivalent residues are defined by determining homology at the level of tertiary and quarternary structure for an acyltransferase whose tertiary and quarternary structure has been determined by x-ray crystallography. In this context, “equivalent residues” are defined as those for which the atomic coordinates of two or more of the main chain atoms of a particular amino acid residue of the carbonyl hydrolase and M. smegmatis acyltransferase (N on N, CA on CA, C on C, and O on O) are within about 0.13 nm and about 0.1 nm after alignment. Alignment is achieved after the best model has been oriented and positioned to give the maximum overlap of atomic coordinates of non-hydrogen protein atoms of the acyltransferase in question to the M. smegmatis acyltransferase. As known in the art, the best model is the crystallographic model giving the lowest R factor for experimental diffraction data at the highest resolution available. Equivalent residues which are functionally and/or structurally analogous to a specific residue of M. smegmatis acyltransferase are defined as those amino acids of the acyltransferase that preferentially adopt a conformation such that they either alter, modify or modulate the protein structure, to effect changes in substrate binding and/or catalysis in a manner defined and attributed to a specific residue of the M. smegmatis acyltransferase. Further, they are those residues of the acyltransferase (in cases where a tertiary structure has been obtained by x-ray crystallography), which occupy an analogous position to the extent that although the main chain atoms of the given residue may not satisfy the criteria of equivalence on the basis of occupying a homologous position, the atomic coordinates of at least two of the side chain atoms of the residue lie within 0.13 nm of the corresponding side chain atoms of M. smegmatis acyltransferase. The coordinates of the three dimensional structure of M. smegmatis acyltransferase were determined and are set forth in Example 14 of WO05/056782 and find use as outlined above to determine equivalent residues on the level of tertiary structure.

Characterization of wild-type and mutant proteins is accomplished via any means suitable and is preferably based on the assessment of properties of interest. For example, pH and/or temperature, as well as detergent and/or oxidative stability is/are determined in some embodiments of the present invention. Indeed, it is contemplated that enzymes having various degrees of stability in one or more of these characteristics (pH, temperature, proteolytic stability, detergent stability, and/or oxidative stability) will find use.

As used herein, “corresponding to,” refers to a residue at the enumerated position in a protein or peptide, or a residue that is analogous, homologous, or equivalent to an enumerated residue in a protein or peptide.

As used herein, “corresponding region,” generally refers to an analogous position along related proteins or a parent protein.

The terms “nucleic acid molecule encoding”, “nucleic acid sequence encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “analogous sequence” refers to a sequence within a protein that provides similar function, tertiary structure, and/or conserved residues as the protein of interest (i.e., typically the original protein of interest). For example, in epitope regions that contain an alpha helix or a beta sheet structure, the replacement amino acids in the analogous sequence maintain the same specific structure. The term also refers to nucleotide sequences, as well as amino acid sequences. In some embodiments, analogous sequences are developed such that the replacement amino acids result in a variant enzyme showing a similar or improved function. In some preferred embodiments, the tertiary structure and/or conserved residues of the amino acids in the protein of interest are located at or near the segment or fragment of interest. Thus, where the segment or fragment of interest contains, for example, an alpha-helix or a beta-sheet structure, the replacement amino acids maintain that specific structure.

As used herein, “homologous protein” refers to a protein (e.g., acyltransferase) that has similar action and/or structure, as a protein of interest (e.g., an acyltransferase from another source). It is not intended that homologs be necessarily related evolutionarily. Thus, it is intended that the term encompass the same or similar enzyme(s) (i.e., in terms of structure and function) obtained from different species. In some preferred embodiments, it is desirable to identify a homolog that has a quaternary, tertiary and/or primary structure similar to the protein of interest, as replacement for the segment or fragment in the protein of interest with an analogous segment from the homolog will reduce the disruptiveness of the change. In some embodiments, homologous proteins induce similar immunological response(s) as a protein of interest.

As used herein, “homologous genes” refers to at least a pair of genes from different species, which genes correspond to each other and which genes are identical or very similar to each other. The term encompasses genes that are separated by speciation (i.e., the development of new species) (e.g., orthologous genes), as well as genes that have been separated by genetic duplication (e.g., paralogous genes). These genes encode “homologous proteins.”

As used herein, “ortholog” and “orthologous genes” refer to genes in different species that have evolved from a common ancestral gene (i.e., a homologous gene) by speciation. Typically, orthologs retain the same function during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, “wild-type”, “native” and “naturally-occurring” proteins are those found in nature. The terms “wild-type sequence,” and “wild-type gene” are used interchangeably herein, to refer to a sequence that is native or naturally occurring in a host cell. The genes encoding the naturally-occurring protein may be obtained in accord with the general methods known to those skilled in the art. The methods generally comprise synthesizing labeled probes having putative sequences encoding regions of the protein of interest, preparing genomic libraries from organisms expressing the protein, and screening the libraries for the gene of interest by hybridization to the probes. Positively hybridizing clones are then mapped and sequenced.

The degree of homology between sequences may be determined using any suitable method known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

As used herein, “percent (%) nucleic acid sequence identity” is defined as the percentage of nucleotide residues in a candidate sequence that are identical with the nucleotide residues of the sequence.

As used herein, the term “hybridization” refers to the process by which a strand of nucleic acid joins with a complementary strand through base pairing, as known in the art.

As used herein, the phrase “hybridization conditions” refers to the conditions under which hybridization reactions are conducted. These conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Alternatively, or in addition, hybridization conditions are based upon the salt or ionic strength conditions of hybridization and/or one or more stringency washes. For example, 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe.

For applications requiring high selectivity, in some embodiments, it is desirable to use relatively stringent conditions to form the hybrids (e.g., relatively low salt and/or high temperature conditions are used).

The phrases “substantially similar” and “substantially identical” in the context of at least two nucleic acids or polypeptides typically means that a polynucleotide or polypeptide comprises a sequence that has at least about 40% identity, at least about 50% identity, at least about 60% identity, at least about 75% identity, at least about 80% identity, at least about 90%, at least about 95%, at least about 97% identity, sometimes as much as about 98% and about 99% sequence identity, compared to the reference (i.e., wild-type) sequence. Sequence identity may be determined using known programs such as BLAST, ALIGN, and CLUSTAL using standard parameters. (See e.g., Altschul, et al., J. Mol. Biol. 215:403-410 [1990]; Henikoff et al., Proc. Natl. Acad. Sci. USA 89:10915 [1989]; Karin et al., Proc. Natl. Acad. Sci. USA 90:5873 [1993]; and Higgins et al., Gene 73:237-244 [1988]). Software for performing BLAST analyses is publicly available through the National Center for Biotechnology Information. Also, databases may be searched using FASTA (Pearson et al., Proc. Natl. Acad. Sci. USA 85:2444-2448 [1988]). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. An indication that two nucleic acid sequences are substantially identical is that the two molecules hybridize to each other under stringent conditions (e.g., within a range of medium to high stringency).

The terms “recovered”, “isolated”, and “separated” as used herein refer to a protein, cell, nucleic acid or amino acid that is removed from at least one component with which it is naturally associated. In certain cases, an isolated protein is a protein that secreted into culture medium and then recovered from that medium.

The term “recombinant” refers to a polynucleotide or polypeptide that does not naturally occur in a host cell. A recombinant molecule may contain two or more naturally-occurring sequences that are linked together in a way that does not occur naturally. A recombinant cell contains a recombinant polynucleotide or polypeptide. Proteins that are produced using recombinant methods are produced using host cells that do not normally produce those proteins.

The term “heterologous” refers to elements that are not normally associated with each other. For example, if a host cell produces a heterologous protein, that protein that is not normally produced in that host cell. Likewise, a promoter that is operably linked to a heterologous coding sequence is a promoter that is operably linked to a coding sequence that it is not usually operably linked to in a wild-type host cell. The term “homologous”, with reference to expression of a polynucleotide or protein, refers to a polynucleotide or protein that occurs naturally in a host cell in which it is expressed.

As used herein, “host cells” are generally prokaryotic or eukaryotic hosts which are transformed or transfected with vectors constructed using recombinant DNA techniques known in the art. Transformed host cells are capable of either replicating vectors encoding the protein variants or expressing the desired protein variant. In the case of vectors which encode the pre- or prepro-form of the protein variant, such variants, when expressed, are typically secreted from the host cell into the host cell medium.

In some embodiments, the present invention pertains to the activity of certain acyltransferases to efficiently catalyze the transfer of an acyl group from an acyl donor, (e.g., a C2 to C20 ester), to an alcohol substrate in an aqueous environment. As described in greater detail herein, in some embodiments, this activity of these enzymes is exploited to make esters that have a pleasant fragrance or flavor. In some other embodiments, the activity of these enzymes is preferably employed to reduce malodor in cleaning applications.

Without any intention to be limited to any particular enzyme, alcohol substrate, or acyl donor, and solely to aid the understanding of some embodiments of the methods described herein, the reaction performed by some embodiments of the subject methods is illustrated below, wherein “AcT” stands for “acyltransferase”.

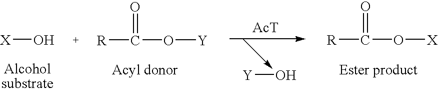

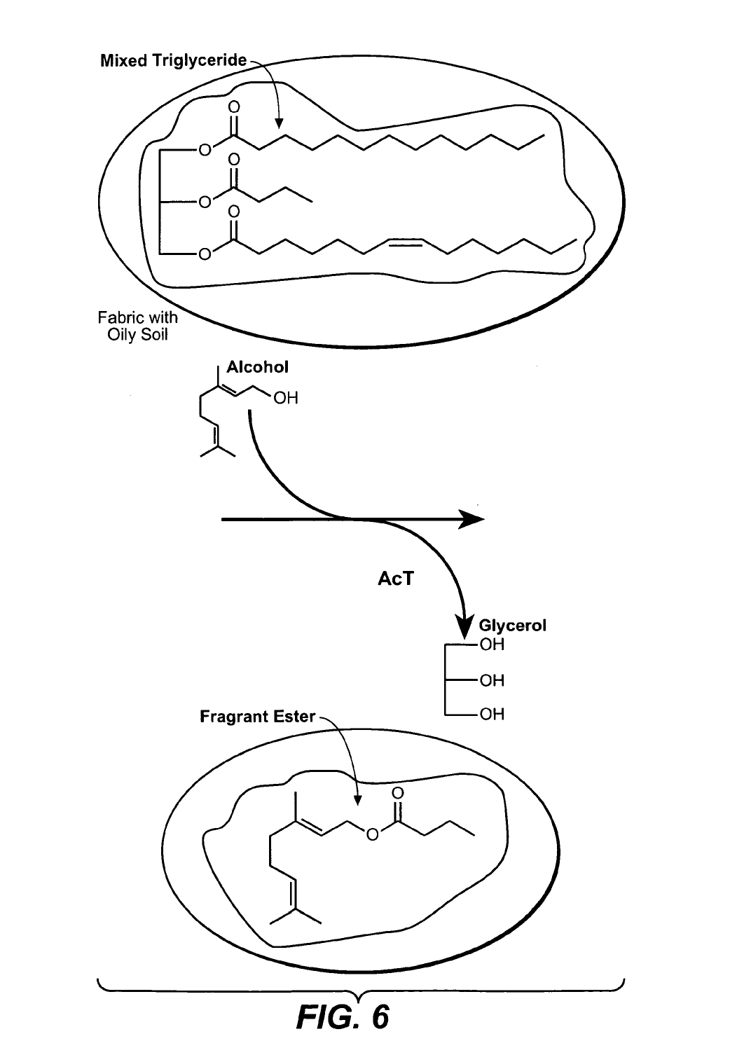

For example, and again without wishing to be limited to any particular enzyme, alcohol substrate, or acyl donor, and solely to aid the understanding of some embodiments of the methods described herein, the acyltransferase enzyme is utilized to transfer an acyl group from a suitable acyl donor (e.g., a triglyceride such as tributyrin or triacetin) to a terpene alcohol such as geraniol or citronellol to produce a fragrant ester. Likewise, in other embodiments, the acyltransferase finds use in reducing the malodor of oily stains. In some particularly preferred embodiments, the oily stains are dairy product stains. In these malodor reduction/prevention embodiments, the AcT enzyme is utilized in order to reduce the amount of foul smelling volatile fatty acids (e.g., butyric acid) produced by hydrolysis of triglycerides. In some embodiments the acyltransferase enzyme synergistically works with at least one lipase enzyme to increase the rate of removal of acyl chains from triacylglyceride, while in other embodiments, the acyltransferase works by linking the acyl chains to an alcohol substrate to produce an ester product, rather than a volatile fatty acid. In some embodiments, the acyltransferase works in both of the above ways. In some embodiments, an acyl chain from the triacylglyceride is linked to an alcohol substrate to produce a fragrant ester. In these embodiments, a fragrant ester, rather than a foul smelling volatile fatty acid, is produced as a byproduct. This embodiment is schematically illustrated in FIG. 6.

These embodiments, as well as many other embodiments, are described in greater detail below.

Prior to the following detailed description, it is noted that the methods discussed herein find use with a variety of different enzymes that have the ability to catalyze the transfer of an acyl group from an acyl donor to an alcohol substrate to produce an ester. Such enzymes include, but are not limited to classical lipases, acyl-CoA-dependent transferases, phospholipases, cutinases, GDSX hydrolases, SGNH hydrolases, serine proteases, and esterases, as well as any enzyme capable of forming an acyl-enzyme intermediate upon contact with an acyl donor, and transferring the acyl group to an acceptor other than water.

In some embodiments, the enzyme is a wild-type enzyme, while in other embodiments, the enzyme has a modified amino acid sequence that causes the enzyme to have altered substrate specificity or increased acyl transferase activity, as compared to the wild-type enzyme. In further describing these embodiments, additional components that find use in; the present invention are provided.

### Acyltransferases

As noted above, the present invention provides ester-producing compositions that contain at least one acyltransferase, and methods of using the enzyme(s). It is contemplated that the acyltransferase of the present compositions comprises any enzyme that can catalyze the transfer of an acyl group from an acyl donor to an alcohol substrate. As noted above, several types of enzymes find use in the methods of the present invention. In some embodiments, the enzyme employed has a higher specificity for alcohol substrates than water. In some of these embodiments, the enzyme exhibits a relative low hydrolysis activity (i.e., a relatively poor ability to hydrolyze an acyl donor in the presence of water) and a relatively high acyltransferase activity (i.e., a better ability to hydrolyze an acyl donor in the presence of an alcohol, in an aqueous environment), wherein the alcoholysis:hydrolysis ratio is greater than about 1.0, a ratio of at least about 1.5, or at least about 2.0. In some embodiments, the acyltransferase also has a higher specificity for peroxide than water, resulting in the production of peracid cleaning agents, (e.g., an perhydrolyis:hydrolysis ratio of greater than about 1.0, a ratio of at least about 1.5, or at least about 2.0).

In some embodiments, a GDSX acyltransferase, in particular a SGNH acyltransferase finds use. Exemplary SGNH acyltransferases that find use in the present invention include the wild-type SGNH acyltransferases deposited in NCBI's GENBANK® database as accession numbers: YP_{—}890535 (GID: 11846860; See also, WO05/056782; M. smegmatis); NP_{—}436338.1 (GID: 16263545; Sinorhizobium meliloti); ZP_{—}01549788.1 (GID: 118592396; Stappia aggregate); NP_{—}066659.1 (GID: 10954724; Agrobacterium rhizogenes); YP_{—}368715.1 (GID: 78065946; Burkholderia sp.); YP_{—}674187.1 (GID: 110633979; Mesorhizobium sp.); and NP_{—}532123.1 (GID: 17935333; Agrobacterium tumefaciens), wild-type orthologs and homologs thereof, and variants thereof that have an amino acid sequence that is at least about 70% identical, at least about 80% identical, at least about 90% identical, at least about 95% identical, or at least at least about 98% identical to any of those wild-type enzymes. These GENBANK® accessions are incorporated by reference in their entirety, including the nucleic acid and protein sequences therein and the annotation of those sequences. Further examples of such enzymes, are obtained by performing sequence homology-based searches of NCBI's GENBANK® database using standard sequence comparison methods known in the art (e.g., BLAST, etc.). In some embodiments, the acyltransferase has an amino acid sequence that is at least about 70% identical to the amino acid sequence set forth in GENBANK® entry YP_{—}890535 (GID: 11846860; M. smegmatis; See also, WO05/056782).

Further exemplary SGNH acyltransferase enzymes include the following, which are referenced by their species and GENBANK® accession numbers: Agrobacterium rhizogenes (Q9KWA6), A. rhizogenes (Q9KWB1), A. tumefaciens (Q8UFG4), A. tumefaciens (Q8UAC0), A. tumefaciens (Q9ZI09), A. tumefaciens (ACA), Prosthecobacter dejongeii (RVM04532), Rhizobium loti (Q98MY5), R. meliloti (Q92XZ1), R. meliloti (Q9EV56), R. rhizogenes (NF006), R. rhizogenes (NF00602875), R. solanacerarum (Q8XQI0), Sinorhizobium meliloti (RSM02162), S. meliloti (RSM05666), Mesorhizobium loti (RML000301), A. rhizogenes (Q9KWA6), A. rhizogenes (Q9KWB1), Agrobacterium tumefaciens (AAD02335), Mesorhizobium loti (Q98MY5), Mesorhizobium loti (ZP00197751), Ralstonia solanacearum (Q8XQI0), Ralstonia eutropha (ZP00166901), Moraxella bovis (AAK53448), Burkholderia cepacia (ZP00216984), Chromobacterium violaceum (Q7NRP5), Pirellula sp. (NP_{—}865746), Vibrio vulnificus (AA007232), Salmonella typhimurium (AAC38796), Sinorhizobium meliloti (SMa1993), Sinorhizobium meliloti (Q92XZ1) and Sinorhizobium meliloti (Q9EV56). The amino acid sequences of these proteins, the sequence alignments, and all other information relating to the above is incorporated by reference herein for all purposes from WO05/056782.

Several examples of such enzymes have been crystallized, and many exemplary amino acid substitutions that are provided for variant enzymes that retain or alter their activity are described in WO05/056782, which is incorporated by reference. Lists of hundreds of amino acid substitutions that are tolerated by and in some embodiments find use in altering the hydrolytic activity, perhydrolytic activity, peracid degradation activity and/or stability of the M. smegmatis perhydrolase are set forth in table 10-3, 10-4, 10-5, 10-6, 10-7, 10-8 and 10-9 of WO05/056782. Given the structural similarity of SGNH acyltransferases, the amino acid substitutions described in WO05/056782 are readily transferable to other members of the SGNH acyltransferase family. Each of the amino acid substitutions described in WO05/056782, and the amino acid sequences produced by those substitutions, is incorporated by reference herein.

In some embodiments, the acyltransferase employed herein is not an acetyl-CoA dependent enzyme. In some alternative embodiments, the GDSX or SGNH acyltransferase used in the instant methods is a wild-type acyltransferase Candida parapsilosis, Aeromonas hydrophila, or Aeromonas salmonicida, while in other embodiments, the acyltransferase is a variant thereof that is at least about 95% identical thereto.

The acyltransferase used in the present invention is produced and isolated using conventional methods, as known in the art. In some embodiments, production of the acyltransferase is accomplished using recombinant methods and a non-native host, which either produces the acyltransferase intracellularly, or secretes the acyltransferase. In some embodiments, a signal sequence is added to the enzyme, which facilitates expression of the enzyme by secretion into the periplasm (i.e., in Gram-negative organisms, such as E. coli), or into the extracellular space (i.e., in Gram-positive organisms, such as Bacillus and Actinomycetes), or eukaryotic hosts (e.g., Trichoderma, Aspergillus, Saccharomyces, and Pichia). It is not intended that any aspect the present invention be limited to these specific hosts, as various other organisms find use as expression hosts in the present invention.

For example, Bacillus cells are well-known as suitable hosts for expression of extracellular proteins (e.g., proteases). Intracellular expression of proteins is less well known. Expression of the enzyme protein intracellularly in Bacillus subtilis is often accomplished using a variety of promoters, including, but not limited to pVeg, pSPAC, pAprE, or pAmyE in the absence of a signal sequence on the 5′ end of the gene. In some embodiments, expression is achieved from a replicating plasmid (high or low copy number), while in alternative embodiments, expression is achieved by integrating the desired construct into the chromosome. Integration is possible at any locus, including but not limited to the aprE, amyE, or pps locus. In some embodiments, the enzyme is expressed from one or more copies of the integrated construct. In alternative embodiments, multiple integrated copies are obtained by the integration of a construct capable of amplification (e.g., linked to an antibiotic cassette and flanked by direct repeat sequences), or by ligation of multiple copies and subsequent integration into the chromosome. In some embodiments, expression of the enzyme with either the replicating plasmid or the integrated construct is monitored using the pNB activity assay in an appropriate culture.

As with Bacillus, in some embodiments, expression of the enzyme in the Gram-positive host Streptomyces is accomplished using a replicating plasmid, while in other embodiments, expression of the enzyme is accomplished via integration of the vector into the Streptomyces chromosome. Any promoter capable of being recognized in Streptomyces finds use in driving transcription of the enzyme gene (e.g., glucose isomerase promoter, A4 promoter). Replicating plasmids, either shuttle vectors or Streptomyces only, also find use in the present invention for expression (e.g., pSECGT).

In other embodiments, the enzyme is produced in other host cells, including but not limited to: fungal host cells (e.g., Pichia sp., Aspergillus sp., or Trichoderma sp. host cells, etc.).

In some embodiments, the enzyme is secreted from the host cell such that the enzyme is recoverable from the culture medium in which the host cell is cultured.

Once it is secreted in to the culture medium, the enzyme is recovered by any suitable and/or convenient method (e.g., by precipitation, centrifugation, affinity, affinity chromatography, ion-exchange chromatography, hydrophobic interaction chromatography two-phase partitioning, ethanol precipitation, reverse phase HPLC, chromatography on silica or on a cation-exchange resin such as DEAE, chromatofocusing, SDS-PAGE, ammonium sulfate precipitation, gel filtration (e.g., Sephadex G-75), filtration or any other method known in the art). Indeed, a number of suitable methods are known to those of skill in the art. In some alternative embodiments, the enzyme is used without purification from the other components of the culture medium. In some of these embodiments, the culture medium is simply concentrated, and then used without further purification of the protein from the components of the growth medium, while in other embodiments it is used without any further modification.

### Alcohol Substrates

Alcohol substrate that find use in the present invention include any organic molecule containing a reactive hydroxyl group that is bound to a carbon atom, excluding hydroxyl-containing polysaccharides and proteins. In some embodiments, the alcohol substrate is of the formula: Z—OH, where Z is any branched, straight chain, cyclic, aromatic or linear organic group, or any substituted version thereof. In some embodiments, Z is a substituted or unsubstituted alkyl, heteroalkyl, alkenyl, alkynyl, aryl, alkylaryl, alkylheteroaryl, or a heteroaryl group containing 2-30 carbon atoms. In some further embodiments, Z is an aliphatic moiety, an aliphatic moiety substituted by an alicyclic or aromatic moiety (e.g., a terpene). In some other embodiments, the alcohol substrate is a polyol, such as a glycol-containing molecule (e.g., tetraethyleneglycol, polyethylene glycol, polypropylene glycol, or polytetrahydrofuran). Suitable alcohol substrates include monomeric polyols (e.g., glycerin), as well as dimeric, trimeric and tetrameric polyols, and sugar alcohols such as erythritol, isomaltitol, lactitol, maltitol, mannitol, sorbitol and xylitol. In some embodiments, polyols are molecules of the formula (Z—OH)n or Z—(OH)n, wherein n is at least about 1, about 2, about 3, about 4, about 5, or about 6 (e.g., where n is 1-4). In some embodiments, the alcohol is present as part of a surfactant or emulsifying agent (e.g., a high linearity primary alcohol such as a NEODOL™ detergent).

In some embodiments, alcohol substrates used in the fragrant ester production methods described below are of the formula Z—OH, where Z is an alicyclic or aromatic moiety, or a terpene, for example.

Exemplary alcohol substrates that find use in the methods of the present invention include, but are not limited to ethanol, methanol, glycerol, propanol, butanol, and the alcohol substrates shown in Tables 1-3 below.

### Acyl Donors

The acyl donor utilized in the methods of the present invention comprises any organic molecule containing a transferable acyl group. In some embodiments, a typical acyl donor is an ester of the formula R¹C(═O)OR², where R¹ and R² are independently any organic moiety, although other molecules also find use. In some embodiments, suitable acyl donors are monomeric, while in other embodiments, they are polymeric, including dimeric, trimeric and higher order polyol esters.

As used herein, a “short chain acyl donor” is an ester of the formula R¹C(═O)OR², where R¹ is any organic moiety that contains a chain of at least 1 to 9 carbon atoms and R² is any organic moiety. In some embodiments, short chain acyl esters contain an acyl chain of 2-10 carbon atoms (i.e., a C₂-C₁₀ carbon chain). Exemplary long chain acyl esters contain a C₆, C₇, C₈, C₉, C₁₀ carbon chain. Exemplary long chain acyl esters contain acetyl, propyl, butyl, pentyl, or hexyl groups, etc.

A “long chain acyl donor” is a ester of the formula R¹C(═O)OR², where R¹ is any organic moiety that contains a chain of at least 10 carbon atoms and R² is any organic moiety. For example, in some embodiments, long chain acyl donors contain a C₁₁, C₁₂, C₁₃, C₁₄, C₁₅, C₁₆, C₁₇, C₁₈, C₁₉, C₂₀, C₂₁, or C₂₂ acyl chain.

Exemplary esters that find use in the present invention include those of the formula:

R¹Oₓ[(R²)ₘ(R³)ₙ]ₚ

wherein R¹ is a moiety selected from the group consisting of H or a substituted or unsubstituted alkyl, heteroalkyl, alkenyl, alkynyl, aryl, alkylaryl, alkylheteroaryl, and heteroaryl. In some embodiments, R¹ comprises from about 1 to about 50,000 carbon atoms, from about 1 to about 10,000 carbon atoms, or even from about 2 to about 100 carbon atoms;

wherein each R² is an optionally substituted alkoxylate moiety (in some embodiments, each R² is independently an ethoxylate, propoxylate or butoxylate moiety);

R³ is an ester-forming moiety having the formula:
R⁴CO— wherein “R⁴” is an H, substituted or unsubstituted alkyl, alkenyl, alkynyl, aryl, alkylaryl, alkylheteroaryl, and heteroaryl (in some embodiments, R⁴ is a substituted or unsubstituted straight or branched chain alkyl, alkenyl, or alkynyl, moiety comprising from 5 to 22 or more carbon atoms, an aryl, alkylaryl, alkylheteroaryl, or heteroaryl moiety comprising from 5 to 12 or more carbon atoms, or R⁴ is a substituted or unsubstituted C₅-C₁₀ or longer alkyl moiety, or R⁴ is a substituted or unsubstituted C₁₁-C₂₂ or longer alkyl moiety);
   x is 1 when R¹ is H; when R¹ is not H, x is an integer that is equal to or less than the number of carbons in R¹;
   p is an integer that is equal to or less than x;
   m is an integer from 0 to 50, an integer from 0 to 18, or an integer from 0 to 12, and n is at least 1.

In some embodiments of the present invention, the molecule comprising an ester moiety is an alkyl ethoxylate or propoxylate having the formula R¹Oₓ[(R²)ₘ(R³)ₙ]ₚ wherein:
R¹ is an C₂-C₃₂ substituted or unsubstituted alkyl or heteroalkyl moiety;
each R² is independently an ethoxylate or propoxylate moiety;
R³ is an ester-forming moiety having the formula:
   R⁴CO— wherein R⁴ is H, substituted or unsubstituted alkyl, alkenyl, alkynyl, aryl, alkylaryl, alkylheteroaryl, and heteroaryl, and in some embodiments, R⁴ is a substituted or unsubstituted straight or branched chain alkyl, alkenyl, or alkynyl moiety comprising from 5 to 22 or more carbon atoms, a substituted or unsubstituted aryl, alkylaryl, alkylheteroaryl, or heteroaryl moiety comprising from 5 to 12 carbon or longer atoms, or R⁴ is a substituted or unsubstituted C₅-C₁₀ or longer alkyl moiety, or R⁴ is a substituted or unsubstituted C₅-C₂₂ or longer alkyl moiety;
   x is an integer that is equal to or less than the number of carbons in R¹;
   p is an integer that is equal to or less than x;
   m is an integer from 1 to 12; and
   n is at least 1.

In some embodiments of the present invention, the molecule comprising the ester moiety has the formula:

R¹Oₓ[(R²)ₘ(R³)ₙ]ₚ

wherein R¹ is H or a moiety that comprises a primary, secondary, tertiary or quaternary amine moiety, said R¹ moiety that comprises an amine moiety being selected from substituted or unsubstituted alkyl, heteroalkyl, alkenyl, alkynyl, aryl, alkylaryl, alkylheteroaryl, and heteroaryl moieties. In some embodiments, R¹ comprises from about 1 to about 50,000 carbon atoms, from about 1 to about 10,000 carbon atoms, or from about 2 to about 100 carbon atoms;

each R² is an alkoxylate moiety (in some embodiments, each R² is independently an ethoxylate, propoxylate or butoxylate moiety);
R³ is an ester-forming moiety having the formula:
   R⁴CO— wherein R⁴ is H, substituted or unsubstituted alkyl, alkenyl, alkynyl, aryl, alkylaryl, alkylheteroaryl, and heteroaryl (in some embodiments, R⁴ is a substituted or unsubstituted straight or branched chain alkyl, alkenyl, or alkynyl moiety comprising from 5 to 22 carbon atoms), a substituted or unsubstituted aryl, alkylaryl, alkylheteroaryl, or heteroaryl moiety comprising from 9 to 12 or more carbon atoms, or R⁴ is a substituted or unsubstituted C₅-C₁₀ or longer alkyl moiety, or R⁴ is a substituted or unsubstituted C₁₁-C₂₂ or longer alkyl moiety;
   x is 1 when R¹ is H; when R¹ is not H, x is an integer that is equal to or less than the number of carbons in R¹;
   p is an integer that is equal to or less than x;
   m is an integer from 0 to 12 or even 1 to 12, and
   n is at least 1.

Suitable acyl donors include triglycerides of any type, including animal-derived triglycerides, dairy-product triglycerides, plant-derived triglycerides and synthetic triglycerides, including, but not limited to triacetin, tributyrin, and longer chain molecules, which provide acetyl groups, butyryl groups, and longer chain acyl groups, respectively. Diacylglycerides, monoacylglycerides, phospholipids, lysophospholipid, glycolipids also find use in the present invention. In some embodiments, diacyl- and triacylglycerides contain the same fatty acid chains, while in other embodiments they contain different fatty acid chains. Other suitable esters include color-forming esters such as p-nitrophenol esters. Additional esters include aliphatic esters (e.g., ethyl butyrate), isoprenoid esters (e.g., citronellyl acetate) and aromatic esters (e.g., benzyl acetate).

In some of the cleaning embodiments of the present invention, the acyl donor is present on an object (e.g., as a stain on the object). In some particularly preferred embodiments, the acyl donor is de-acylated by the subject composition in situ.

In some embodiments, some of the fragrant ester-production methods described in greater detail herein require transfer of short chain (e.g., C2-C10) acyl groups such as acetyl, and butyryl groups.

### Cleaning Compositions

The present invention also provides cleaning compositions comprising at least one acyltransferase and at least one alcohol substrate for the acyltransferase. In some embodiments, the cleaning composition is formulated to clean objects stained with an acyl donor molecule (e.g., a triglyceride) in situ. Thus, in some embodiments, the acyltransferase and alcohol substrate are present in amounts effective to produce a detectable ester upon contact of the cleaning composition with an acyl donor-containing object. In some embodiments, the cleaning composition, upon contact with an acyl donor-containing object, further comprises the acyl donor-containing object, and an ester that is produced as result of a reaction, catalyzed by the acyltransferase, between the alcohol substrate and the acyl donor. As noted above, in some embodiments, the acyltransferase is an SGNH acyltransferase. In some additional embodiments, the cleaning composition contains an alcohol substrate and acyl donor combination such that when the acyl group from the acyl donor is transferred to the alcohol substrate by the acyltransferase, a fabric care agent (e.g., a surfactant ester) is produced.

In some embodiments, the alcohol substrate is a dual-purpose molecule in that it also functions as a surfactant or emulsifying agent present in the cleaning composition. Examples of such alcohol substrates include, but are not limited to: fatty alcohols (e.g., C8-C18 linear or branched aliphatic alcohols), for example cetyl alcohol (e.g., hexadecan-1-ol), fatty alcohol ethoxylates (e.g. NEODOL™ ethoxylates) derived from fatty alcohols, and polyol ethoxylates (e.g., glycerin ethoxylates) which are commonly employed in cleaning compositions.

As described in greater detail below, the cleaning compositions of the present invention are provided in any suitable form, including solids (e.g., with the enzyme and alcohol substrate adsorbed onto a solid material), liquids, and gels. In some preferred embodiments, the compositions are provided in concentrated form. In other embodiments, the subject cleaning composition are employed as is, and in some further embodiments are used as a spray or pre-wash composition. In use, the working form of the cleaning composition (e.g., the dissolved or diluted form of the cleaning composition) is aqueous and thus contains at least about 50% water, and in many cases contains between about 50% and about 99.99% water. In some embodiments, the working concentration of alcohol substrate in a subject cleaning composition is from about 0.0001% to about 50% (v/v or w/v), less than about 1%, less than about 0.1%, less than about 0.01%, or less than about 0.001% alcohol. In some embodiments, the working concentration of the subject acyltransferase enzyme in the cleaning composition is about 0.01 ppm (parts per million, w/v) to about 1000 ppm, about 0.01 ppm to about 0.05 ppm, about 0.05 ppm to about 0.1 ppm, about 0.1 ppm to about 0.5 ppm, about 0.5 ppm to about 1 ppm, about 1 ppm to about 5 ppm, about 5 ppm to about 10 ppm, about 10 ppm to about 50 ppm, about 50 ppm to about 100 ppm, about 100 ppm to about 500 ppm, or about 500 ppm to about 1000 ppm.

In some embodiments, the cleaning compositions of the present invention further comprise at least one lipase (e.g., a triacylglycerol lipase having an activity defined as EC 3.1.1.3, according to IUBMB enzyme nomenclature). In some embodiments, the lipase is a classical lipase, as described above. It is contemplated that the acyltransferase and the lipase act synergistically to remove acyl chains from acylglyceride molecules (e.g., triacylglycerol) on an object. However, it is not intended that the present invention be limited to any particular mechanism of action.

In some embodiments, the cleaning composition comprises a source of peroxide, which can be hydrogen peroxide itself or a composition that produces hydrogen peroxide as a reaction product. Suitable hydrogen peroxide sources that produce hydrogen peroxide as a reaction product include, but are not limited to peroxygen sources selected from: (i) from about 0.01 to about 50, from about 0.1 to about 20, or from about 1 to 10 weight percent of a per-salt, an organic peroxyacid, urea hydrogen peroxide and mixtures thereof; (ii) from about 0.01 to about 50, from about 0.1 to about 20, or from about 1 to 10 weight percent of a carbohydrate and from about 0.0001 to about 1, from about 0.001 to about 0.5, from about 0.01 to about 0.1 weight percent carbohydrate oxidase; and (iii) mixtures thereof. Suitable per-salts include, but are not limited to alkalimetal perborate, alkalimetal percarbonate, alkalimetal perphosphates, alkalimetal persulphates and mixtures thereof.

In some embodiments, the saccharide is selected from monosaccharides, disaccharides, trisaccharides, oligosaccharides (e.g., carbohydrates), and mixtures thereof. Suitable saccharides include, but are not limited to saccharides selected from D-arabinose, L-arabinose, D-cellobiose, 2-deoxy-D-galactose, 2-deoxy-D-ribose, D-fructose, L-fucose, D-galactose, D-glucose, D-glycero-D-gulo-heptose, D-lactose, D-lyxose, L-lyxose, D-maltose, D-mannose, melezitose, L-melibiose, palatinose, D-raffinose, L-rhamnose, D-ribose, L-sorbose, stachyose, sucrose, D-trehalose, D-xylose, L-xylose, and mixtures thereof.

Suitable carbohydrate oxidases include, but are not limited to carbohydrate oxidases selected from aldose oxidase (IUPAC classification EC1.1.3.9), galactose oxidase (IUPAC classification EC1.1.3.9), cellobiose oxidase (IUPAC classification EC1.1.3.25), pyranose oxidase (IUPAC classification EC1.1.3.10), sorbose oxidase (IUPAC classification EC1.1.3.11) and/or hexose oxidase (IUPAC classification EC1.1.3.5), glucose oxidase (IUPAC classification EC1.1.3.4), and mixtures thereof.

In some embodiments, the acyl donor-containing object cleaned by the cleaning composition is stained with an oily substance (e.g., a substance containing triacylglyceride or the like). In some embodiments, the object (e.g., a fabric), is stained with a dairy product.

While not essential for the performance of the methods described below, in some embodiments the choice of alcohol substrate is chosen to produce a fragrant ester upon reaction with the acyl donor. Fragrant esters are described in greater detail below.

In some embodiments, the cleaning composition is a fabric cleaning composition (i.e., a laundry detergent), a surface cleaning composition, or a dish cleaning composition, or an automatic dishwasher detergent composition. Formulations for exemplary cleaning compositions are described in great detail in WO0001826, which is incorporated by reference herein.

In a some embodiment, the subject cleaning composition contain from about 1% to about 80%, about 5% to about 50% (by weight) of at least one surfactant (e.g., non-ionic surfactants, cationic surfactants, anionic surfactants, or zwitterionic surfactants, or any mixture thereof). Exemplary surfactants include, but are not limited to alkyl benzene sulfonate (ABS), including linear alkyl benzene sulfonate and linear alkyl sodium sulfonate, alkyl phenoxy polyethoxy ethanol (e.g., nonyl phenoxy ethoxylate or nonyl phenol), diethanolamine, triethanolamine, and monoethanolamine. Exemplary surfactants that find use in detergents, particularly laundry detergents, include those described in U.S. Pat. Nos. 3,664,961, 3,919,678, 4,222,905, and 4,239,659.

In some embodiments, the detergent is a solid, while in other embodiments it is liquid, and in other embodiments it is a gel. In some preferred embodiments the detergents further comprise a buffer (e.g., sodium carbonate, or sodium bicarbonate), detergent builder(s), bleach, bleach activator(s), additional enzyme(s), enzyme stabilizing agent(s), suds booster(s), suppressor(s), anti-tarnish agent(s), anti-corrosion agent(s), soil suspending agent(s), soil release agent(s), germicide(s), pH adjusting agent(s), non-builder alkalinity source(s), chelating agent(s), organic or inorganic filler(s), solvent(s), hydrotrope(s), optical brightener(s), dye(s), and/or perfumes.

In some embodiments, the subject cleaning composition comprises one or more other enzymes (e.g., pectin lyases, endoglycosidases, hemicellulases, peroxidases, proteases, cellulases, xylanases, lipases, phospholipases, esterases, cutinases, pectinases, pectate lyases, amylases, mannanases, keratinases, reductases, oxidases, oxidoreductases, phenoloxidases, lipoxygenases, ligninases, pullulanases, tannases, pentosanases, malanases, beta-glucanases, arabinosidases, hyaluronidase, chondroitinase, laccase, and amylases) or mixtures thereof. In some embodiments, a combination of enzymes (i.e., a “cocktail”) comprising conventional applicable enzymes like protease, lipase, cutinase and/or cellulase in conjunction with acyltransferase is used.

A wide variety of other ingredients useful in detergent cleaning compositions are also provided in the compositions herein, including other active ingredients, carriers, hydrotropes, processing aids, dyes or pigments, solvents for liquid formulations, etc. In embodiments in which an additional increment of sudsing is desired, suds boosters such as the C₁₀-C₁₆ alkolamides are incorporated into the compositions, typically at about 1% to about 10% levels.

In some embodiments, detergent compositions contain water and other solvents as carriers. Low molecular weight primary or secondary alcohols exemplified by methanol, ethanol, propanol, and isopropanol are suitable. Monohydric alcohols are preferred for solubilizing surfactants, but polyols such as those containing from about 2 to about 6 carbon atoms and from about 2 to about 6 hydroxy groups (e.g., 1,3-propanediol, ethylene glycol, glycerine, and 1,2-propanediol) also find use. In some embodiments, the compositions contain from about 5% to about 90%, typically from about 10% to about 50% of such carriers.

In some embodiments, the detergent compositions provided herein are formulated such that during use in aqueous cleaning operations, the wash water has a pH between about 6.8 and about 11.0. Thus, finished products are typically formulated at this range. Techniques for controlling pH at recommended usage levels include the use of buffers, alkalis, acids, etc., and are well known to those skilled in the art. In some embodiments, the cleaning composition comprises an automatic dishwashing detergent that has a working pH in the range of about pH 9.0 to about pH 11.5, about pH 9.0 to about pH 9.5, about pH 9.5 to about pH 10.0, about pH 10.0 to about pH 10.5, about pH 10.5 to about pH 11.0, or about pH 11.0 to about pH 11.5. In some other embodiments, the cleaning composition comprises a liquid laundry detergent that has a working pH in the range of about pH 7.5 to about pH 8.5, about pH 7.5 to about pH 8.0, or about pH 8.0 to about pH 8.5. In some other embodiments, the cleaning composition comprises a solid laundry detergent that has a working pH in the range of about pH 9.5 to about pH 10.5, about pH 9.5 to about pH 10.0, or about pH 10.0 to about pH 10.5.

Various bleaching compounds, such as the percarbonates, perborates and the like, also find use in the compositions of the present invention, typically at levels from about 1% to about 15% by weight. As desired, such compositions also contain bleach activators such as tetraacetyl ethylenediamine, nonanoyloxybenzene sulfonate, and the like, which are also known in the art. Usage levels typically range from about 1% to about 10% by weight.

Various soil release agents, especially of the anionic oligoester type, various chelating agents, especially the aminophosphonates and ethylenediaminedisuccinates, various clay soil removal agents, especially ethoxylated tetraethylene pentamine, various dispersing agents, especially polyacrylates and polyasparatates, various brighteners, especially anionic brighteners, various suds suppressors, especially silicones and secondary alcohols, various fabric softeners, especially smectite clays, and the like, all find use in various embodiments of the present compositions at levels ranging from about 1% to about 35% by weight. Standard formularies are well-known to those skilled in the art.

Enzyme stabilizers also find use in some embodiments of the present cleaning compositions. Such stabilizers include, but are not limited to propylene glycol (preferably from about 1% to about 10%), sodium formate (preferably from about 0.1% to about 1%), and calcium formate (preferably from about 0.1% to about 1%).

In still further embodiments, the cleaning compositions of the present invention also comprise at least one builder. In some preferred embodiments, builders are present in the compositions at levels from about 5% to about 50% by weight. Typical builders include the 1-10 micron zeolites, polycarboxylates such as citrate and oxydisuccinates, layered silicates, phosphates, and the like. Other conventional builders are listed in standard formularies and are well-known to those of skill in the art.

Other optional ingredients include chelating agents, clay soil removal/anti redeposition agents, polymeric dispersing agents, bleaches, brighteners, suds suppressors, solvents and aesthetic agents.

The present invention also provides methods for the use of the cleaning compositions provided herein. In some embodiments, the cleaning methods include: combining at least one acyltransferase, at least one alcohol substrate for the acyltransferase, and an object soiled with an acyl donor-containing substance; wherein the acyltransferase catalyzes transfer of an acyl group from the acyl donor onto the alcohol substrate to produce an ester. In some embodiments, the alcohol substrate is chosen so as to produce a resultant fragrant ester. In some other embodiments, the acyl group is transferred to a surfactant or emulsifying agent, or one or more of the other agents listed above. In some embodiments, the cleaning composition further comprises an acyl donor that serves no other cleaning function (i.e., does not serve as a surfactant, emulsifier, oxidizer, etc.,) other than to produce fragrance. Such acyl donors include, but are not limited to triacetin and tributyrin.

In some alternative embodiments, the cleaning methods of the present invention include the step of producing an ester that has cleaning properties, such as an ester surfactant or ester emulsifying agent, that has a cleaning activity during the wash.

In some embodiments, the object may be a fabric (including, but not limited to clothing, upholstery, carpet, bedding, etc.), or a hard surface (including but not limited to kitchen surfaces, bathroom surfaces, tiles, etc), or dishware. In some embodiments, the fabric is soiled with an oil-containing substance such as a triacylglyceride-containing substance. In some embodiments, the oil-containing substance comprises at least one C4-C18 triacylglyceride (e.g., dairy products).

In some embodiments, the cleaning methods utilize a cleaning composition that contains acetyl transferase but does not contain a lipase (e.g., a classical lipase). In some alternative embodiments, the subject cleaning methods utilize cleaning composition that contain the subject acetyltransferase and a lipase (e.g., Lipolase™, Lipozym™, Lipomax™, Lipex™, Amano™ lipase, Toyo-Jozo™ lipase, Meito™ lipase or Diosynth™). In some embodiments, use of a particular an acyltransferase-lipase combination results in significantly less malodor than if the method is performed using the lipase enzyme alone. It is not intended that the present invention be limited to any particular mechanism or theory. However, it is contemplated that the acyltransferase and lipase work synergistically to remove acyl groups from triacylglyceride (e.g., butyric acid-containing triacylglyceride), to reduce malodor.

Therefore, in some embodiments, use of an acyltransferase in a cleaning composition results in more than about a 10% reduction in malodor-causing fatty acids, about a 20% reduction in malodor-causing fatty acids, more than about a 30% reduction in malodor-causing fatty acids, more than about a 50% reduction in malodor-causing fatty acids, more than about a 70% reduction in malodor-causing fatty acids, more than about an 80% reduction in malodor-causing fatty acids, or more than about a 90% reduction in malodor-causing fatty acids; as compared to equivalent cleaning compositions that do not contain the acyltransferase. In some particularly preferred embodiments, use of a subject acyltransferase in a cleaning composition produces no malodor.

### Compositions for Production of Fragrant Esters

As noted above, the present invention provides compositions and methods for the production of fragrant esters. In some embodiments, the composition comprises at least one acyltransferase, an alcohol substrate for the acyltransferase, and an acyl donor. In some of these embodiments, the acyltransferase catalyzes transfer of an acyl group from the acyl donor to the alcohol substrate to produce a fragrant ester in an aqueous environment. In some embodiments, this composition is a substantially dry (e.g., dehydrated) composition in which fragrant ester is only produced upon rehydration of the composition. In other embodiments, the composition is an aqueous composition that further comprises the fragrant ester.

In many embodiments, the alcohol substrate and the acyl donor of the composition are chosen to produce a particular fragrant ester. Exemplary fragrant esters that are produced using the subject composition are set forth in Tables 1-3 below, along with a suitable combination of alcohol substrate and acyl donor for the production of those esters. Other fragrant esters are known, and given the molecular structure of such fragrant esters, the alcohol substrate and ester that can be combined in the presence of a subject acyltransferase would be apparent. In these Tables, “AcT” is the wild type acyltransferase of M. smegmatis, “KLM3′” is the acyltransferase of Aeromonas sp., as described in WO04/064987, and Lipomax™ is a lipase from Pseudomonas alcaligenes (Genencor).

TABLE 1
Transesterification of Aliphatic Alcohols

| Alcohol | Structure | Ester | Acyl Donor | Enzyme |
|---|---|---|---|---|
| Ethanol | 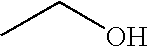 | Butyrate | Tributyrin, p-NB butterfat | AcT, KLM3′ Lipomax |
| 2-methyl-butan-1-ol | 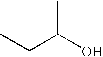 | Acetate Butyrate | Triacetin, p-NB, tributyrin | AcT, KLM3′ |
| 3-methyl-butan-1-ol | 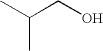 | Acetate Butyrate | Triacetin, tributyrin | AcT |
| Hexyl alcohol | 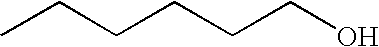 | Acetate | Triacetin, tributyrin | AcT |
| cis-3-hexen-1-ol | 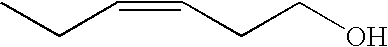 | Acetate Butyrate | Triacetin, tributyrin | AcT, KLM3′ |
| Cyclohexylmethanol | 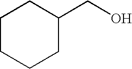 | Acetate | Triacetin | AcT |
| Cyclohexylethanol | 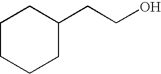 | Acetate | Triacetin | AcT |

TABLE 2
Transesterification of Terpene Alcohols

| | | | Acyl | |
|---|---|---|---|---|
| Alcohol | Structure | Ester | Donor | Enzyme |
| Geraniol | 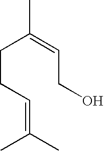 | Acetate Butyrate | Triacetin, tributyrin | AcT |
| Citronellol | 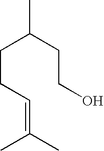 | Acetate Butyrate | Triacetin, tributyrin | AcT, KLM3′ |
| Nerol | 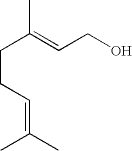 | Acetate | Triacetin | AcT |
| Myrtenol | 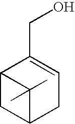 | Acetate | Triacetin | AcT |
| Myrtanol | 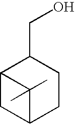 | Acetate | Triacetin | AcT |

TABLE 3
Transesterification of Aromatic Alcohols

| Alcohol | Structure | Ester | Acyl Donor | Enzyme |
|---|---|---|---|---|
| Benzyl alcohol | 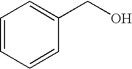 | Acetate Butyrate | Triacetin butterfat | AcT |
| Phenethyl alcohol | 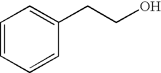 | Acetate | Triacetin | AcT, KLM3′ |
| Piperonyl alcohol | 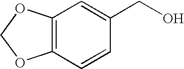 | Acetate | Triacetin | AcT |
| Veratryl alcohol | 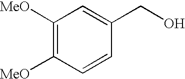 | Acetate | Triacetin | AcT |

In some embodiments, the SGNH acyltransferase is immobilized on a substrate, (e.g., a solid or semi-solid support) such as a column or gel to allow the reaction to be terminated by washing the alcohol substrate and acyl donor from the enzyme.

### Methods for Production of Fragrant Esters

The above-described composition find use in a variety of fragrant ester-producing methods that generally involve combining at least one acyltransferase, at least one alcohol substrate for the acyltransferase, and at least one acyl donor, where, in an aqueous environment, the acyltransferase catalyzes transfer of an acyl group from the acyl donor onto the alcohol substrate to produce the fragrant ester. In some embodiments, the methods involve rehydrating the components after they are combined. In some alternative embodiments, the acyltransferase, the alcohol substrate and the acyl donor are combined in an aqueous environment. As noted above, in some alternative embodiments, the acyltransferase is an SGNH acyltransferase.

These methods find utility in a variety of processes in which fragrant esters are desirable. For example, in some embodiments, the composition is incorporated into foodstuffs to improve or produce flavors or fragrance during consumption, or used in cleaning methods, as described above. In some further embodiments, the compositions are used in ester manufacturing methods.

In one example, the fragrant ester-producing composition is incorporated in dried form (e.g., adsorbed onto a substrate), into a foodstuff such as chewing gum or candy. Rehydration of the foodstuff (e.g., during mastication or by the addition of water-containing liquid such as water or milk), initiates the acyltransferase reaction to produce the fragrant ester in situ. Likewise, in some embodiments, the methods are used to make bulk fragrant esters for the food, perfume and/or cleaning industries.

In some embodiments, the alcohol substrate is itself be a fragrant alcohol. As such, in some embodiments, the odor of the reaction described above changes over time, (e.g., from the odor of the fragrant alcohol substrate to the odor of an ester of that alcohol).

In some further embodiments, a fragrant alcohol is transesterified using a long acyl chain (e.g., a long chain fatty acid) to produce a non-fragrant ester. In some of these embodiments, the non-fragrant ester is hydrolyzed over time, spontaneously, or in the presence of a hydrolase, to reproduce the fragrant alcohol.

Methods for Production of Surfactant Esters in situ

In some embodiments, in situ modification of lipids is carried out using particles containing an acyltransferase, phospholipids and sorbitol. In some embodiments, the particles are comprise forms produced by nanoencapsulation, microencapsulation, tablet-making, pelleting, and/or by using coatings of WAX ester, as specified in the “Bariere System” known to those of skill in the art.

In some embodiments, further coating is provided by the temperature protection technology (TPT) system. In some embodiments, the concentrations of both the lipid substrate, phospholipid and the acceptor molecule, sorbitol are very low in the washing process and thereby limit the production of green detergent. In some embodiments, by including the substrate and the acceptor molecules together with the KLM3 enzyme in a closed compartment assure that the concentration of reactants are high enough for a fast bioconversion process. In some embodiments, during storage at specified conditions of temperature and moisture, KLM3 catalyzes an in situ modification process and thereby create lyso-PC and sorbitol-acyl esters. To allow a complete conversion of the phospholipids (PC) the ratio between PC and sorbitol is optimized to a ratio of about 1:2; about 1:5, about 1:10, about 1:50, or most preferably about 1:100 for PC:sorbitol. In some embodiments, to accommodate the best detergent composition, all of the phospholipids are converted to the lyso-phospholipid derivatives and the equivalent amount of sorbitol-acyl esters. With the optimal KLM3 acyltransferase mutant the enzymatic reaction only gives rise to lyso-phospholipids and sorbitol-acyl ester, without significant amounts of free fatty acids. To achieve a powerful effect of the detergent, all of the phospholipids are converted to the lyso-phopholipid derivatives.

In some embodiments, the biochemical reaction takes place after the encapsulation and in some embodiments requires additional shelf time. When the reaction is completed, the particles are added to the washing powder. The particles are solubilized during the washing process and the detergents are released. A large range of both substrates (triglycerides, diglyceridesmonoglycerides, phospholipids, galactolipids, vinylesters, methyl esters etc. of fatty acids) find use. Similarly, a large number of acceptor molecules are also suitable. These acceptors comprise sorbitol, xylitol, glucose, maltose, sucrose, polyols, and long, medium and short chain alcohols, polysaccharides, such as pectin, starch, galactomannan, alginate, carageenans chitosan, hydrolysed chitosan and oligosaccharides derived from these polysaccharides. In additional embodiments, acceptor molecules are polypeptides and peptides.

The complete disclosure of WO05/056782 including but not limited to all descriptions of acyltransferase enzymes, amino acid alterations, crystal structures, assay methods, methods of use, sequences, homologs, orthologs, sequence alignments, figures, tables, cleaning compositions, etc., is incorporated by reference herein for all purposes.

### EXPERIMENTAL

The following examples are provided in order to demonstrate and further illustrate certain preferred embodiments and aspects of the present invention and are not to be construed as limiting the scope thereof.

PCT publication WO05/056782 relates to the identification and use of acyltransferase enzymes. Each of examples 1-27 of PCT publication WO05/056782 is individually incorporated by reference herein for disclosure of all methods disclosed therein including but not limited to disclosure of: methods of making acyltransferases, methods of identifying acyltransferases, methods of testing acyltransferases, acyltransferases polynucleotide and polypeptide sequences, methods of using acyltransferases and compositions in which acyltransferases may be employed.

### Example 1

### Acylation of cis-3-Hexenol, 2-Phenylethanol and Isoamyl Alcohol

Acylation of cis-3-hexenol, 2-phenylethanol and isoamyl alcohol was performed in water with tributyrin and a soluble acyltransferase.

In a typical procedure, the alcohol (2 uL) and tributyrin (2 uL) in 200 mM phosphate buffer, pH 7 (500 uL) were treated with acyltransferase (M. smegmatis; AcT) (34 ppm) or KLM3′ (20 ppm) at 45° C. for 40 min. Dichloromethane (500 uL) was then added to each vial, followed by vortex agitation (10 seconds) and centrifugation to separate the organic and aqueous layers. The organic layer was then removed and analyzed by GC/MS. This analysis was conducted with an Agilent 6890 GC/MS using a 30 m×0.25 mm (0.25 um film) HP-5MS column. The GC/MS method utilized helium as the carrier gas (lcc/min) with an injector port temperature of 250° C. and a 20:1 split ratio. The oven temperature program began with a 1 min hold at 60° C., increasing to 300° C. at 30° C./min for a total run time of 10 minutes. Mass detector was initiated at 2 min post injection scanning from 30 to 400 AMU.

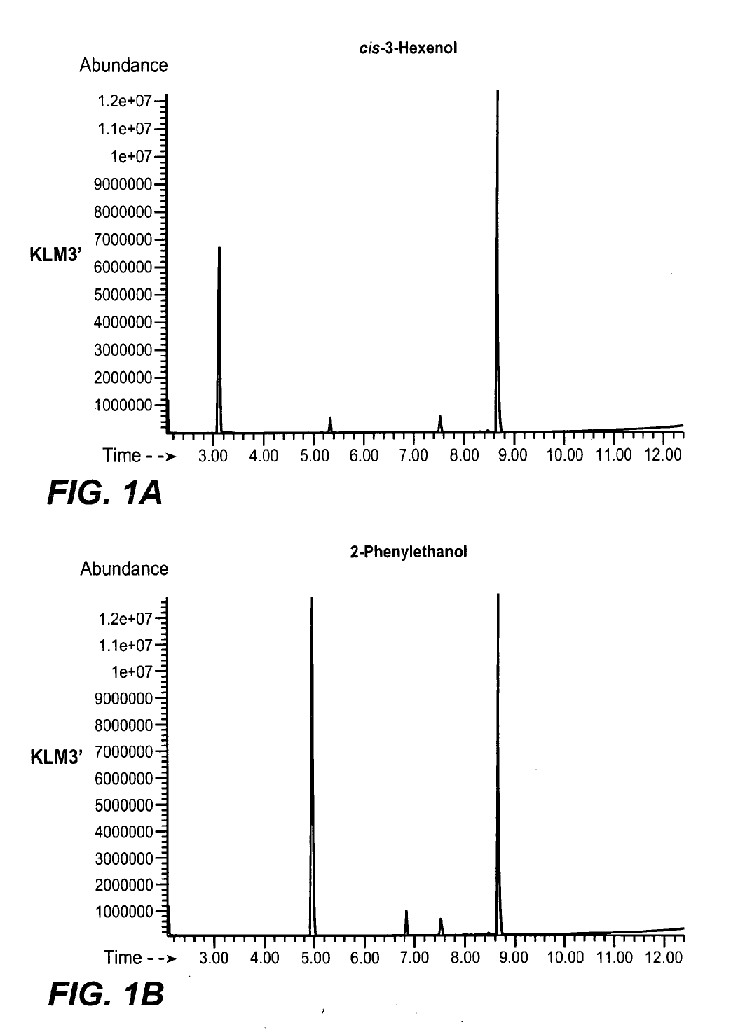

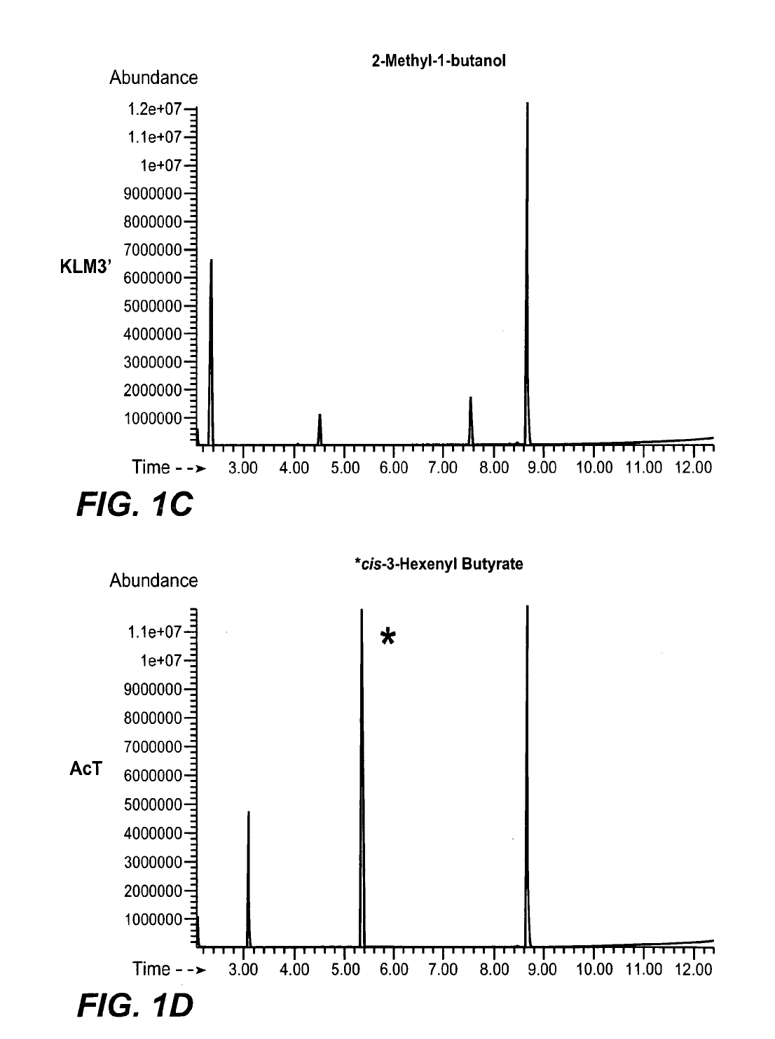

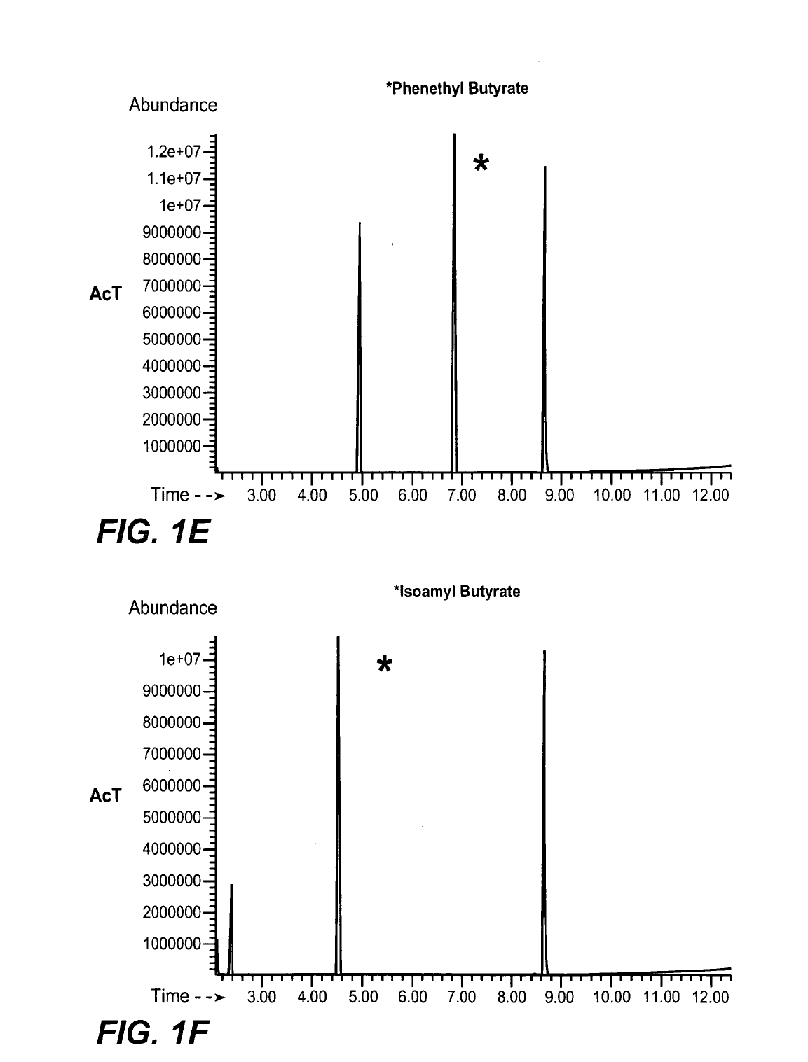

FIG. 1 indicates that in each of these experiments a proportion of the alcohol was converted to their respective butyric acid esters. This amount was significantly greater for AcT than for KLM3′.

### Example 2

### Acylation of Citronellol and Geraniol

The terpene alcohols citronellol (1) and geraniol (2) were assessed as substrates for the acyltransferases AcT and KLM3′ using both triacetin and tributyrin as acyl donors.

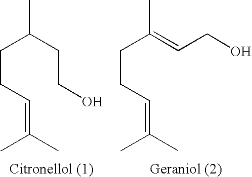

Terpene alcohols (2 uL) and either triacetin or tributyrin (2 uL) in 50 mM phosphate buffer, pH 7 (500 uL) were treated with AcT (34 ppm) or KLM3′ (20 ppm) at 45° C. for 40 min. An aliquot (50 uL) was then removed from each reaction, diluted into methanol/dichloromethane (1:3, 500 uL) and analyzed by GC/MS for evidence of ester production. The results are provided in Table 4.

TABLE 4
Extent of Conversion of Citronellol and Geraniol to Their Respective
Acetyl and Butyryl Esters with Two Acyltransferases.

| | |
|---|---|
| Citronellol | Geraniol |

| Enzyme | Acetate | Butyrate | Acetate | Butyrate |
|---|---|---|---|---|
| AcT | ++ | +++ | +++ | ++++ |
| KLM3′ | Trace | Trace | None | None |

### Example 3

### Acylation of Alcohols in Water with An Acyltransferase Adsorbed on Fabric I

An 1 mL aliquot of an acyltransferase solution (100 ppm in 5 mM HEPES buffer, pH 8) was added to the center of a square section of knit cotton cloth (10×10 cm) and the cloth allowed to air-dry overnight.

Aliquots (10 mL) of a solution containing benzyl alcohol (1% v/v) and triacetin (1% v/v) in 50 mM sodium phosphate buffer, pH 7, were added to both the cloth swatch with adsorbed AcT, as well as a no enzyme control. The characteristic odor of benzyl acetate was generated within 2 minutes on the fabric containing the AcT enzyme, in contrast to the control, which produced no noticeable odor.

### Example 4

### Acylation of Alcohols in Water with an Acyltransferase Immobilized onto Fabric II

Knit cotton fabric swatches (20 by 20 cm) were placed on a plastic sheet and treated with AcT (1 ml of 12 mg/mL), polyethylenimine (500 uL of a 20% w/v solution) and deionized water (1 mL). The fabric was allowed to dry overnight under ambient conditions after which time it was removed from the plastic sheet and soaked in 50 mM sodium phosphate buffer (400 mL, pH 7) with slow stirring overnight. The cotton swatch was then rinsed thoroughly with tap water and allowed to drip dry. A second cotton swatch was prepared according to the method described above except that the enzyme mixture applied to the fabric contained a latex suspension (1 mL of AIRFLEX™ 423, AirProducts, Allentown, Pa.) in addition to the components listed above.

The two swatches were placed side by side and treated with an aqueous solution of benzyl alcohol (2% v/v) and triacetin (2% v/v) in 50 mM sodium phosphate buffer (40 mL of pH 7). The odor of benzyl acetate was clearly evident from both swatches, in contrast to a control swatch. The swatches were also treated with a solution of p-nitrophenyl butyrate (200 uL of 10 mM in water) in order to visualize the hydrolytic activity of the bound AcT. In this case the cotton swatch treated with AcT/PEI only gave a noticeable color.

### Example 5

### Acylation of Benzyl Alcohol in Water by Rehydration of a Triacetin/Alcohol Mixture Adsorbed on Starch

Benzyl alcohol (0.5 mL) and triacetin (0.5 mL) were added to 10 g of maltodextrin (Grain Processing Corp., IA) followed by vigorous mechanical agitation resulting in a free-flowing powder with little or no odor. A portion of this mixture (1 g) was placed in a Petri dish and was then treated with a solution of AcT (1 ppm) resulting in the production of the characteristic odor of benzyl acetate in under 5 minutes. A control was performed using water and did not result in the production of benzyl acetate in under 1 hour.

### Example 6

### Transesterification Using AcT Immobilized in a Silica Sol-Gel

Acyltransferase (AcT) was immobilized in a silica sol gel and compared to the soluble form of the enzyme for the ability to produce fragrant esters under aqueous conditions.

### i) Sol Gel Encapsulation of AcT

An aliquot (2.2 mL) of a 1:1 mixture of sodium silicate (27% SiO₂, 14% NaOH, Sigma Aldrich Corp., WI) and sodium methyl siliconate (30% in water, Gelest, N.J.) was added to phosphoric acid (4 mL of 1.5 M) with stirring. A solution of acyltransferase (1 mL of 12 mg/mL) was then added and the mixture and allowed to stand at room temperature until gelation ensued. The resulting gel was then washed twice with 50 mM phosphate buffer, pH 7 (50 mL) and cured overnight in a sealed container.

ii) Esterification of cis-3-hexenol

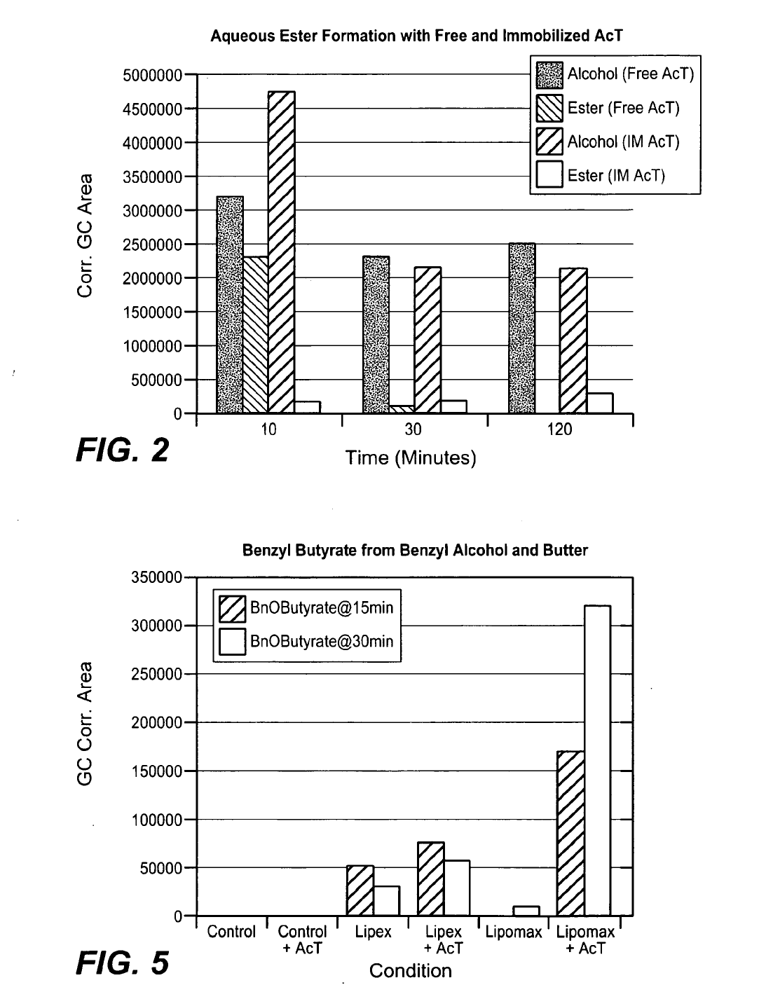

A portion of the wet sol-gel (0.66 g, equivalent to 1 mg of AcT) described above was incubated with cis-3-hexenol (20 uL) and triacetin (40 uL) in 50 mM sodium phosphate buffer, pH 7. The conversion of the cis-3-hexenol to the acetyl ester was compared to a control containing soluble AcT (0.5 mg of AcT). Aliquots (10 uL) were taken from the two reactions at 10, 30 and 120 minutes and were analyzed by GC/MS. The results are shown in FIG. 2.

While it is clear that the immobilized enzyme forms the acetyl ester (retention time 4.5 minutes) at a lower rate than the free enzyme, removal of the immobilized form of the enzyme prevents the subsequent hydrolysis of the fragrant ester, as is apparent for the free enzyme at the and 120 minute time points.

### Example 7

### Transesterfication of an Alcohol and a Fragrant Ester Using AcT

A mixture of benzyl alcohol and citronellyl acetate (1% v/v each) in 50 mM K phosphate buffer, pH 7 was treated with AcT (10 ppm) at room temperature. Within several minutes the characteristic odors of benzyl acetate and citronellol became apparent. The presence of these compounds was confirmed by GC/MS using the method described in Example 1. The results of the experiment demonstrated the possibility of producing two fragrances simultaneously from precursors with less pronounced odors.

### Example 8

### Fragrant Ester Production from Butter-Soiled Fabric

Molten butter (40-50 mg) was applied to 6 knit woven cotton swatches (250-300 mg each) and allowed to cool to room temperature. The swatches were weighed and then treated with either LIPOMAX or AcT or combinations of the two enzymes (Table 5). Each swatch was added to 20 mL of 5 mM HEPES buffer, pH 7 containing benzyl alcohol (10 uL, 0.005% v/v) and the enzyme(s). Following agitation at room temperature for 20 minutes the swatches were removed and assessed for odor before and after drying by two panelists. The total loss in weight was also measured following drying. The results are summarized in Table 6.

TABLE 5
Extent of Butter Removal from Butter-soiled
Cotton Fabric Swatches

| | | Fabric wt | Butter wt | Butter wt | |
|---|---|---|---|---|---|
| Swatch | Condition | (mg) | before | after | % Loss |
| 1 | Control | 276.6 | 40.7 | 39.0 | 4.2% |
| 2 | 1 ppm AcT | 280.5 | 45.1 | 37.9 | 16% |
| | 1 ppm LM | | | | |
| 3 | 1 ppm AcT only | 289.5 | 46.6 | 42.8 | 8% |
| 4 | 1 ppm LM only | 271.7 | 45.3 | 38.3 | 15% |
| 5 | 2 ppm AcT | 275.4 | 45.9 | 39.4 | 14% |
| | 2 ppm LM | | | | |
| 6 | 1 ppm AcT | 261.1 | 47.3 | 40.7 | 14% |
| | 5 ppm LM | | | | |

AcT = M. smegmatis acyltransferases; LM = LIPOMAX ™ lipase.

TABLE 6
Extent of Malodor Formation of Butter-Soiled
Swatches After Enzyme Treatment

| Swatch | Wet Odor (n = 2) | Dry Odor (n = 1) | Dry odor description |
|---|---|---|---|
| 1 | 0 | −0.5 | Trace rancid |
| 2 | +2 | −2 | Strong rancid |
| 3 | +0.5 | −0.5 | Trace rancid |
| 4 | −1 5 | −2 | Strong rancid |
| 5 | +2 | −1 | Trace fruity/rancid |
| 6 | +1.5 | 0 | Fruity/rancid |

### Example 9

### Determination of the Ratio of Transesterification Versus Hydrolysis

Tributyrin (10 uL) was added to buffer (1 mL) containing 4% ethanol and treated with either AcT or KLM3′, plus an enzyme-free control at 40° C. over 2 h. An aliquot (100 uL) was removed from each sample and diluted into dichloromethane (900 uL), followed GC/MS analysis. The amount and ratio of ethyl butyrate to butyric acid was noted for each condition. The control showed no acyltransfer or hydrolysis of the substrate. The AcT treated sample showed a complete digestion of the tributyrin, and a butyric acid to ethyl butyrate ratio of 1:2. The KLM3′ treated sample showed only partial digestion of the tributyrin, however the butyric acid to ethyl butyrate ratio was 1:5.

### Example 10

### Simultaneous Production of a Peracid and a Fragrance Achieved Using Both Soluble and Immobilized Forms of AcT

The combination of AcT, triacetin, dilute aqueous hydrogen peroxide (50 to 500 ppm) and benzyl alcohol (10-50 ppm) results in the production of both peracetic acid and the fragrant benzyl acetate.

A solution of benzyl alcohol (50 uL), glycerol triacetate (triacetin, 100 uL) and the dye pinacyanol chloride (50 uL of 1 mg/mL in 80% acetone) was treated with 30% hydrogen peroxide (100 uL) and a 75 ppm solution of acyltransferase (100 uL). The characteristic fragrance of benzyl alcohol was detected in 1 to 2 minutes. The dye was completely decolorized within 10 minutes. The unpleasant odor of peracetic acid was substantially masked by the fragrance.

The experiment was repeated with cyclohexylmethanol (50 uL) and resulted in the bleaching of the dye and the formation of fragrant cyclohexylmethyl acetate. A control experiment in which AcT was omitted did not result in significant fragrance formation or dye bleaching.

A solution of acyltransferase (1 mL of 10 ppm) was added to a small knit cotton swatch (5×5 cm) and allowed to dry. Addition of 1-2 mL of solution of benzyl alcohol (50 uL), glycerol triacetate (triacetin, 100 uL), 30% hydrogen peroxide (100 uL) and the dye pinacyanol chloride (50 uL of 1 mg/mL in 80% acetone) resulted in the generation of fragrant benzyl acetate and the bleaching of the dye.

The order of addition could be reversed whereby 1-2 mL of a solution of benzyl alcohol (50 uL), glycerol triacetate (triacetin, 100 uL) and the dye pinacyanol chloride (50 uL of 1 mg/mL in 80% acetone) was added to the fabric swatch and allowed to dry. Subsequent addition of AcT (1 mL of 10 ppm) and hydrogen peroxide (1 mL of 3%) resulted in the bleaching of the dye from purple to colorless and the odor of benzyl acetate within 10 minutes.

### Example 11

### Acylation of Polyols with Tributyrin in a Detergent Background

A) An emulsion of tributyrin (1% v/v) and tetraethyleneglycol (1% v/v) in 5 mM HEPES buffer, pH 7.8 containing 1.5 g/L AATCC HDL was prepared by thorough vortex mixing. An aliquot (200 uL) of this mixture was diluted 10-fold by addition to 1.8 mL of 5 mM HEPES buffer, pH 7.8 containing 1.5 g/L AATCC HDL and treated with AcT (10 ppm) at room temperature with stirring. Small aliquots (50 uL) were withdrawn at defined timepoints and diluted into 20% aqueous acetonitrile followed by LC/MS analysis.

LC/MS analysis was performed on a Surveyor HPLC system interfaced to a Quantum TSQ triple quadrupole mass spectrometer (ThermoFisher, San Jose, Calif.) operating in positive electrospray (+ve ESI) mode. The HPLC column used was an Agilent Zorbax SB-Aq C18 column (100×2.1 mm). Compounds were eluted using a gradient beginning with Solvent A (25 mM ammonium formate in H₂O) with increasing amounts of Solvent B (90% methanol+10% solvent A), returning to solvent A over 10 minutes.

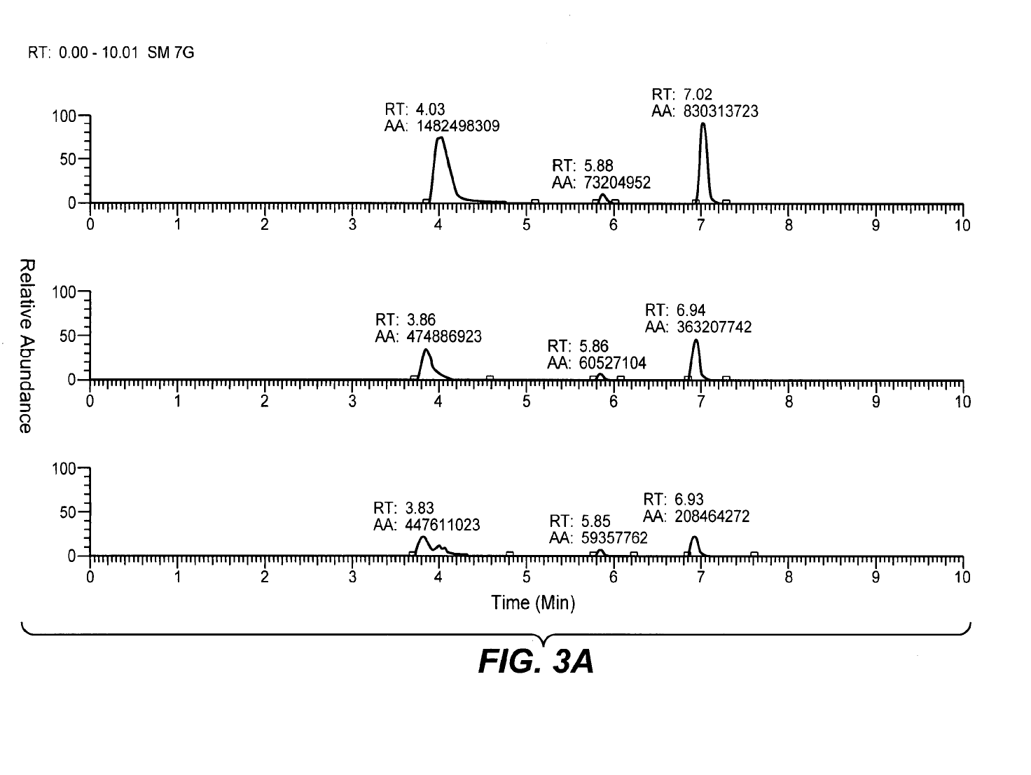

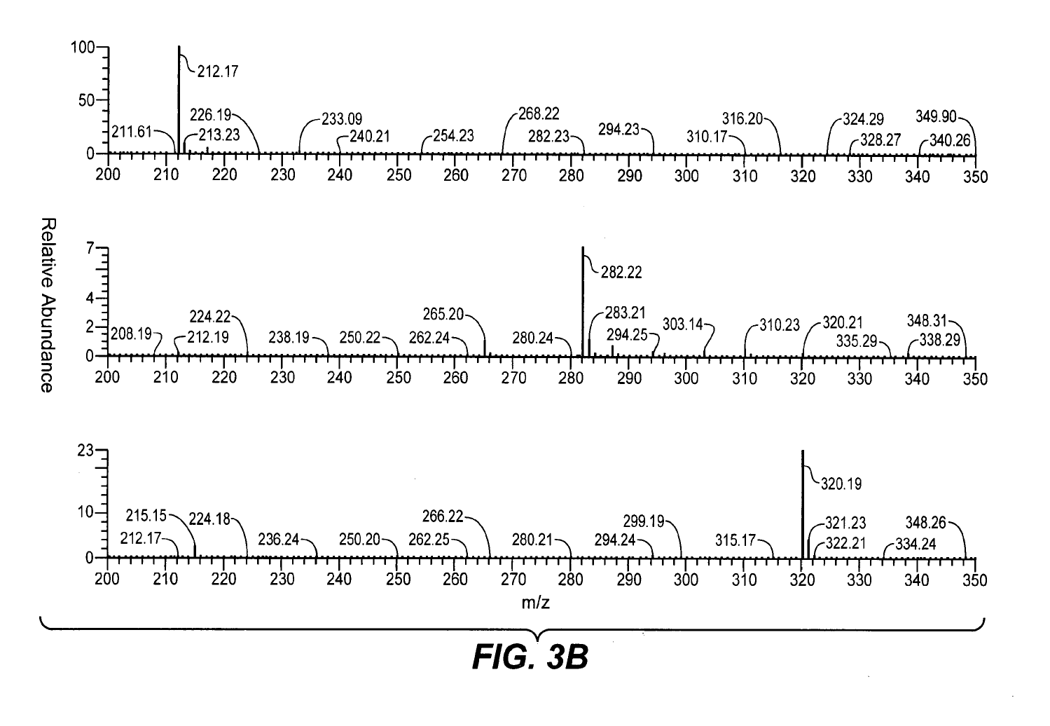

Initially only the two starting materials were observed, tetraethyleneglycol eluting at 3.9 minutes with m/z of 212 and tributyrin at 6.9 minutes with a m/z of 320. Both compounds gave the expected m/z ratios for their ammonium ion adducts. Following the addition of the AcT enzyme, a new peak was observed eluting at 5.8 minutes with a m/z of 282, corresponding to the monobutyryl ester of tetraethylene glycol (FIG. 3). After overnight stirring, the odor of butyric acid was clearly apparent.

B) The above experiment was repeated using ¹³C-uniformly labeled glycerol (¹³C-U-glycerol) and tributyrin. The isotopically-labeled substrate allowed discrimination between glycerol (m/z 110), monobutyrin (m/z 180) and dibutyrin (m/z 250) derived from the tributyrin acyl donor, from the butyrate esters (m/z 183 and 253 for mono- and dibutyrin respectively) formed by acylation of the labeled glycerol acyl acceptor (m/z 113).

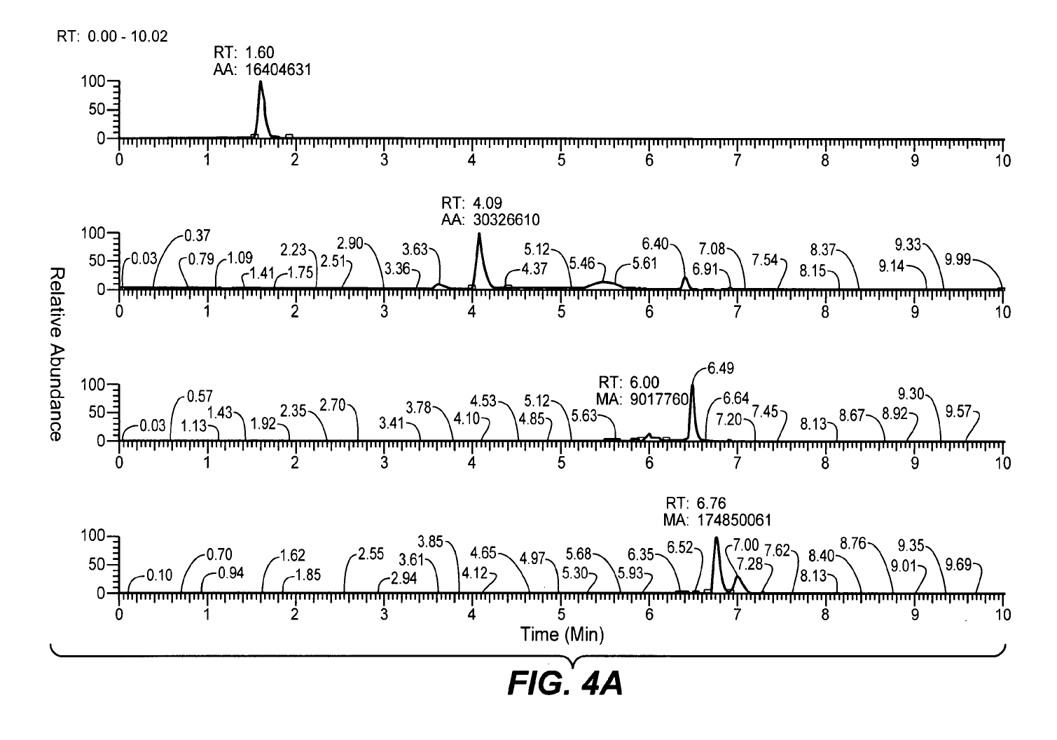

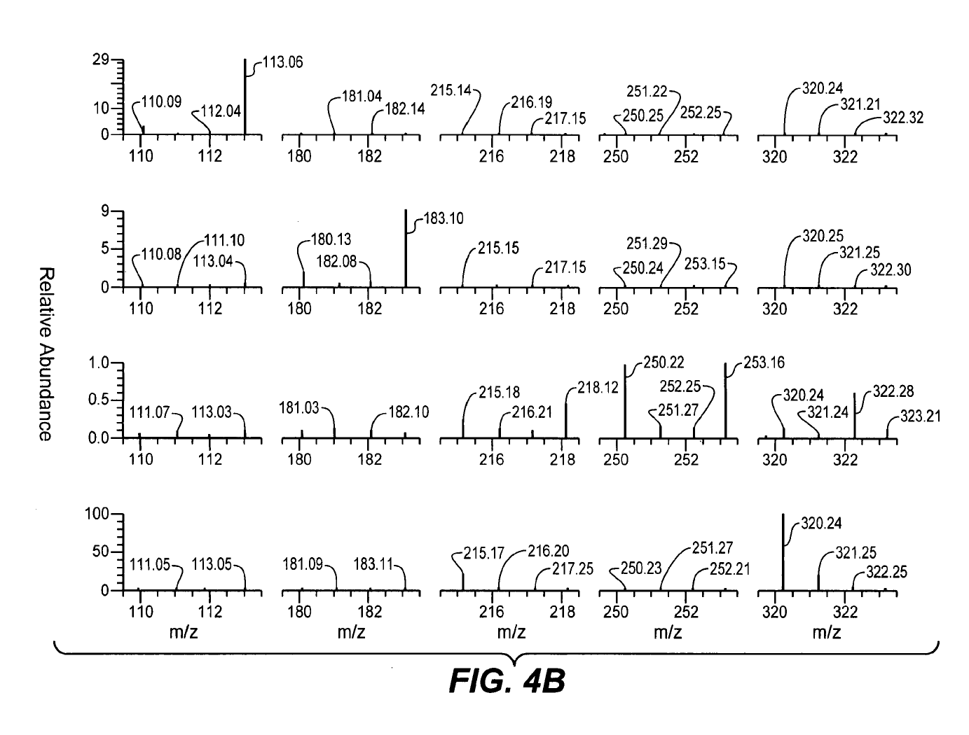

LC/MS analysis (FIG. 4) of the mixture following overnight incubation shows the formation of labeled mono- and dibutyrin, in addition to the unlabeled analogs.

### Example 12

### Fragrance Generation from Butterfat-Soiled Fabric Under Laundry Conditions

Butterfat-soiled cotton swatches were washed under laundry conditions in a Terg-O-tometer (U.S. Testing, Co. Inc. Hoboken, N.J.) in the presence of a lipase and/or Acyltransferase (AcT) plus an acceptor alcohol with the aim of both reducing the amount of free short chain fatty acids (C4 to C8) and the creation of pleasant smelling short chain fatty acid esters.

Butterfat soiled swatches (CFT CS-10, Test Fabrics, Inc. West Pittston, Pa., USA) (6 per 1 L Terg pot) were treated with either no lipase, Lipex (Novozymes)(1.2 ppm) or Lipomax (Genencor) (2 ppm) plus or minus Acyltransferase (AcT) (2 ppm) in a heavy duty liquid detergent (AATCC HDL) background (1.5 g/L) in 5 mM HEPES buffer, pH 7.8, hardness 6 gpg. Benzyl alcohol (1 g/L) was added to each pot prior to the 30 minute wash period at 77° F.

At both the 15 and 30 minute timepoints, an aliquot (8 mL) was taken from each pot and extracted with hexane (2 mL). The hexane layer was separated from the aqueous emulsion in a centrifuge and 1 mL added to gas chromatography (GC) vials. GC/MS analysis was conducted with an Agilent 6890 GC/MS using a 30 m×0.25 mm (0.25 um film) HP-5MS column. The GC/MS method utilized helium as the carrier gas (1 cc/min) with an injector port temperature of 250° C. and a 20:1 split ratio. The oven temperature program began with a 1 min hold at 60° C., increasing to 240° C. at 20° C./min for a total run time of 10 minutes. Mass detector was initiated at 2 min post injection scanning from 30 to 400 AMU.

The GC/MS results are shown in FIG. 5 and below in Table 7. No benzyl butyrate was detected in either the control (pot 1) or the control+AcT (pot 2) pots. Both Lipex and Lipomax alone produced some benzyl butyrate with the former producing more at both timepoints. The addition of AcT enhanced the amount of benzyl butyrate produced for both lipases, but the effect was far greater for Lipomax, suggesting a strong synergistic effect.

TABLE 7
Benzyl Butyrate Formation From Butterfat-Soiled
Cotton Under Laundry Conditions

Benzyl butyrate (GC corr. area)

| Condition | 15 minutes | 30 minutes |
|---|---|---|
| Blank | 0 | 0 |
| Control | 0 | 0 |
| Control + AcT | 0 | 0 |
| Lipex | 53000 | 32000 |
| Lipex + AcT | 77000 | 59000 |
| Lipomax | 0 | 11000 |
| Lipomax + AcT | 170000 | 320000 |

### Example 13

### Reduction of Malodor from Butterfat-Soiled Fabric Under Laundry Conditions

Following the washing experiment described in Example 12, the cotton swatches were dried overnight and assessed subjectively for malodor, summarized in Table 8.

TABLE 8
Assessment of Malodor on Butter-Soiled
Cotton Following Washing

| Pot # | Condition | Comments |
|---|---|---|
| 1 | Control | Buttery/neutral |
| 2 | Control + AcT | Buttery/neutral |
| 3 | Lipex | Rank/Foul odor |
| 4 | Lipex + AcT | Rank/Foul odor |
| 5 | Lipomax | Unpleasant, but less so than swatches from |
| | | pots #3 and #4 |
| 6 | Lipomax + AcT | Slightly off odor, less unpleasant than #5 |

The worst malodor was associated with the Lipex treated swatches. Lipomax treated swatches were significantly less foul, although worse than control. There was a noticeable reduction in malodor in the Lipomax plus AcT treated swatches, relative to Lipomax only.

### Example 14

### Use of KLM3′ to Make Sorbitol Monooleate from Sorbitol and Egg Yolk

Lipid acyl transferase KLM3 mutant pLA231 was tested by incubation in a system containing egg yolk and sorbitol for 4 hours at 40° C.

The reaction product was extracted with organic solvent and the isolated lipids were analyzed by HPTLC and GLC/MS. The results confirm the ability of KLM3 mutant pLA 231 to produce sorbitol monooleate from sorbitol and egg yolk.

In the detergent industry it is known to use sorbitol in different formulations. It is also known that fabrics often contain fatty stains including fats/oils and eggs.

One purpose of this investigation was to study the effect of a KLM3 mutant in a mixture of sorbitol and egg yolk with the aim to produce a surfactant for cleaning purposes.

### Materials and Methods.

KLM3 variant pLA231: mutation W122A, A236E, L31F (activity: 1.6 TIPU/ml)

Sorbitol, 70% (Danisco)

Egg yolk: Pasteurized egg yolk from Hedegaard, DK 9560 Hadsund.

Sorbitol monooleate reference component identified from Grindsted SMO item no. 452454

### HPTLC

Applicator: CAMAG applicator AST4.

HPTLC plate: 20×10 cm (Merck no. 1.05641)

The plate was activated before use by drying in an oven at 160° C. for 20-30 minutes.

Application: 8.0 μl of extracted lipids dissolved in Chloroform:Methanol (2:1) was applied to the HPTLC plate using AST4 applicator.

Running-buffer:4: Chloroform:Methanol:Water (74:26:4)

Application/Elution time: 16 minutes.

Developing fluid: 6% Cupriacetate in 16% H₃PO₄

After elution, the plate was dried in an oven at 160° C. for 10 minutes, cooled and immersed in the developing fluid and then dried additional in 6 minutes at 160° C. The plate was evaluated visually and scanned (Camag TLC scanner).

### GLC Analysis

Perkin Elmer Autosystem 9000 Capillary Gas Chromatograph equipped with WCOT fused silica column 12.5 m×0.25 mm ID×0.1μ film thickness 5% phenyl-methyl-silicone (CP Sil 8 CB from Chrompack).

Carrier gas: Helium.

Injector. PSSI cold split injection (initial temp 50° C. heated to 385° C.), volume 1.0 μl

Detector FID: 395° C.

| Oven program: | 1 | 2 | 3 |
|---|---|---|---|
| Oven temperature, ° C. | 90 | 280 | 350 |
| Isohtermal, time, min. | 1 | 0 | 10 |
| Temperature rate, ° C./min. | 15 | 4 | |

Sample preparation: Lipid extracted from samples were dissolved in 0.5 ml Heptane:Pyridin, 2:1 containing internal standard heptadecane, 0.5 mg/ml. 300 μl sample solution was transferred to a crimp vial, 300 μl MSTFA (N-Methyl-N-trimethylsilyl-trifluoraceamid) was added and reacted for 20 minutes at 60° C.

### Experimental

KLM3 pLA 231 was tested in a substrate of egg yolk and sorbitol according to the recipe shown in Table 9.

TABLE 9

| Jour. 2467-112 | | 1 | 2 |
|---|---|---|---|
| Egg yolk | g | 0.67 | 0.67 |
| Sorbitol, 70% | g | 0.33 | 0.33 |
| KLM3, pLA 231, 1 TIPU/ml | ml | 0.1 | |
| water | ml | | 0.1 |

### Procedure

Egg yolk and sorbitol was mixed with magnetic stirrer in a dram glass and heated to 50° C. The enzyme was added and incubated for 4 hours at 50° C.

The reaction was stopped by adding 7.5 ml Chloroform:Methanol 2.1 and mixing on a Whirley. The lipids were extracted on a Rotamix (25 rpm) for 30 minutes and the samples were centrifuged at 700 g for 10 minutes. 1 ml of the solvent phase was taken out for TLC and GLC/MS analysis.

### Results

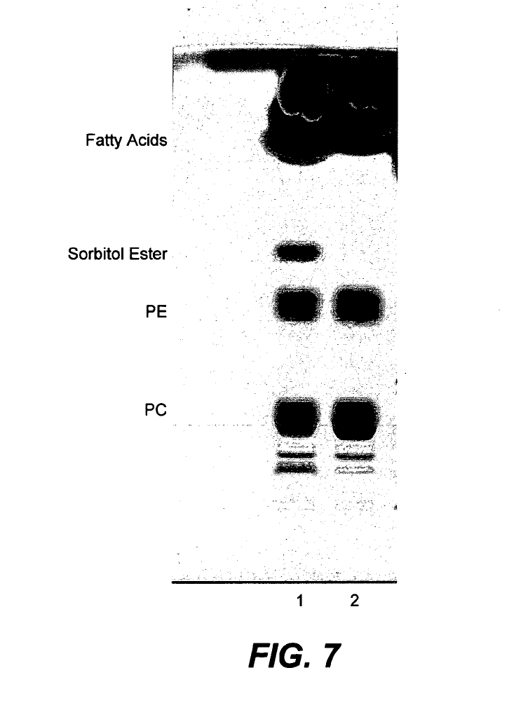

The HPTLC analysis of the lipids from samples 1 and 2 are shown in FIG. 7.

The HPTLC chromatogram indicate the formation of a polar component which is expected to be sorbitol ester.

For further identification the samples were analyzed by GLC/MS

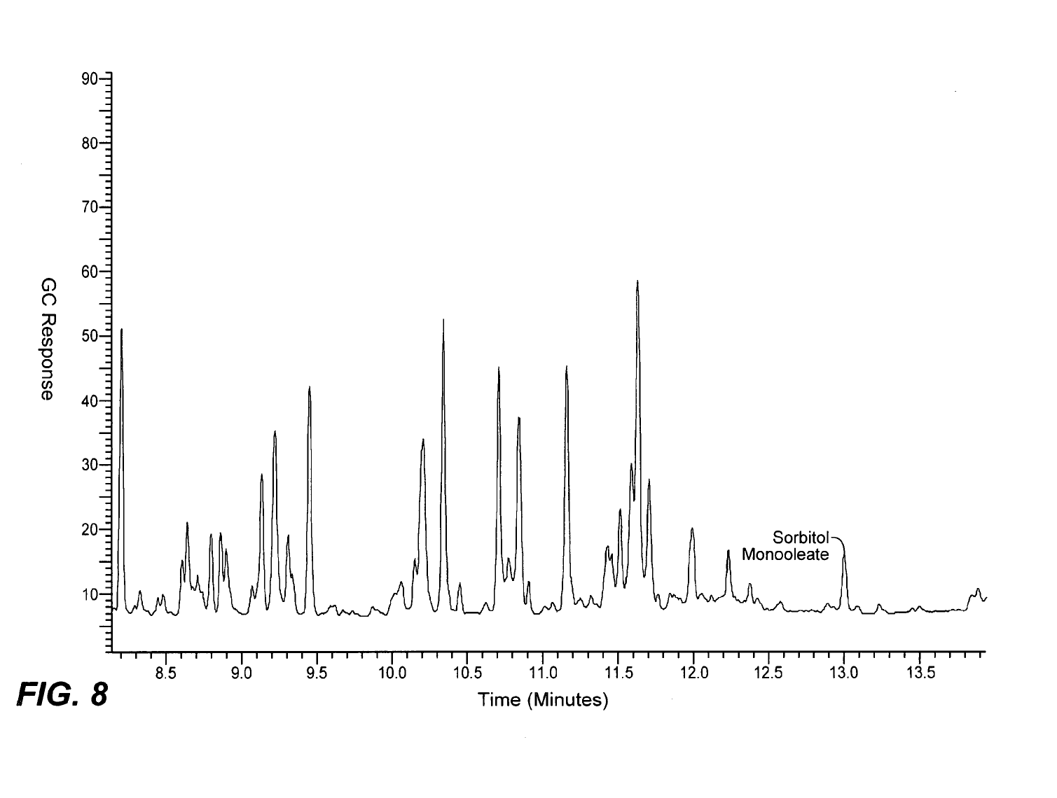

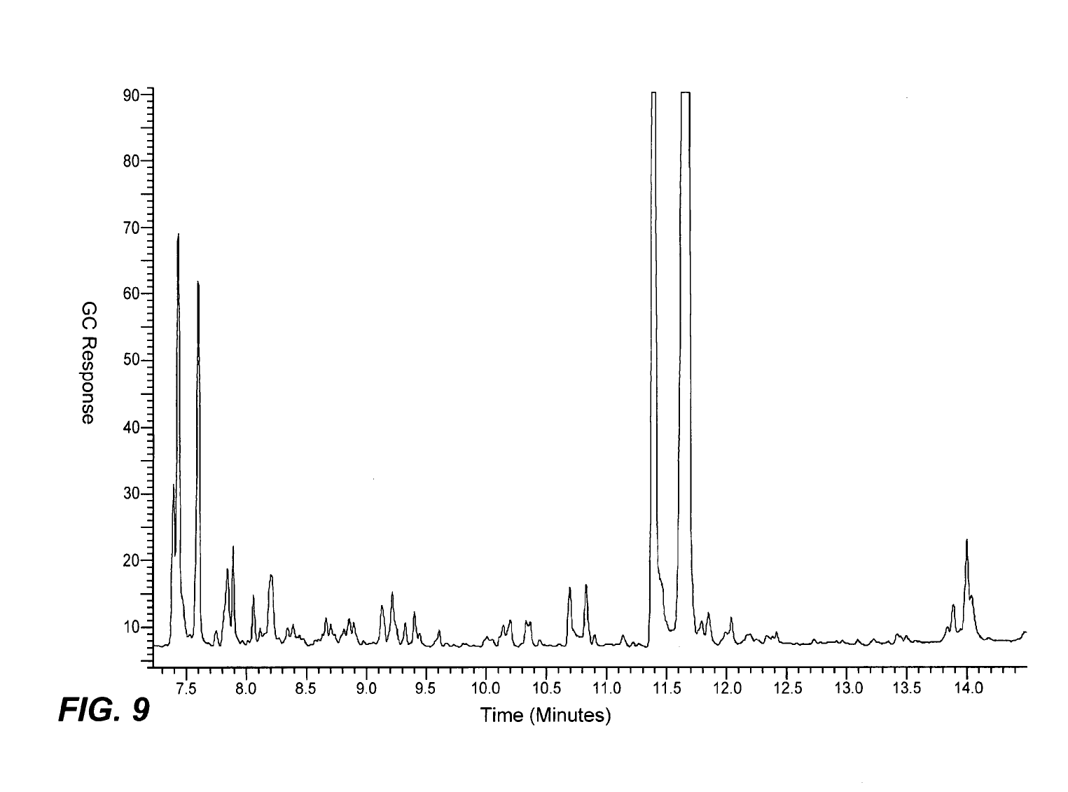

The GLC chromatogram of enzyme treated sample (1) and Control sample (2) are shown in FIGS. 8 and 9.

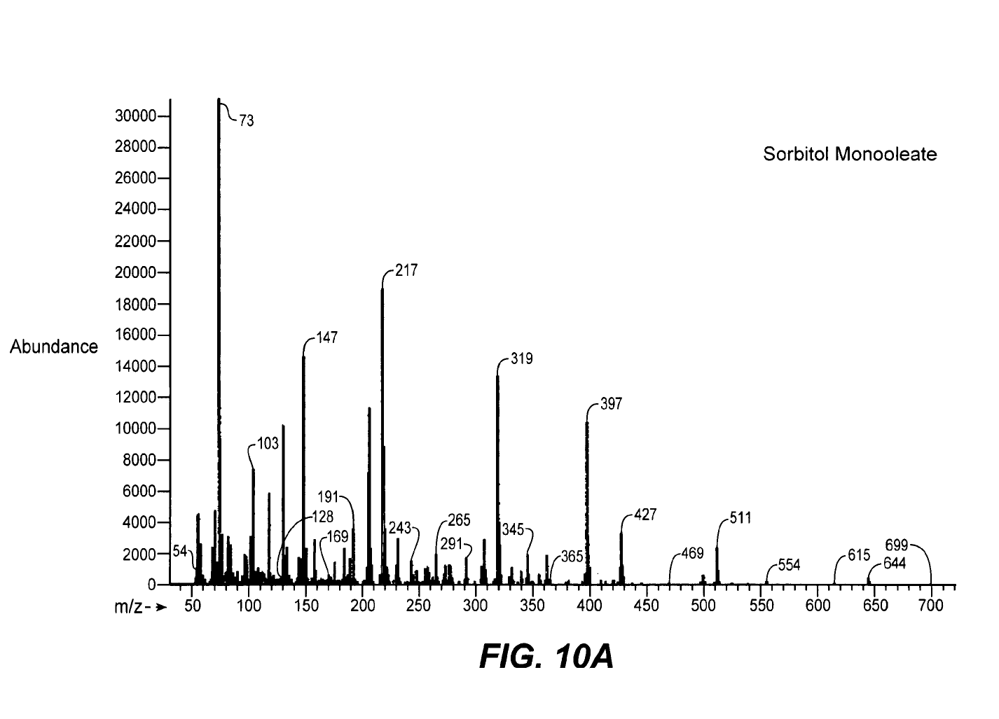

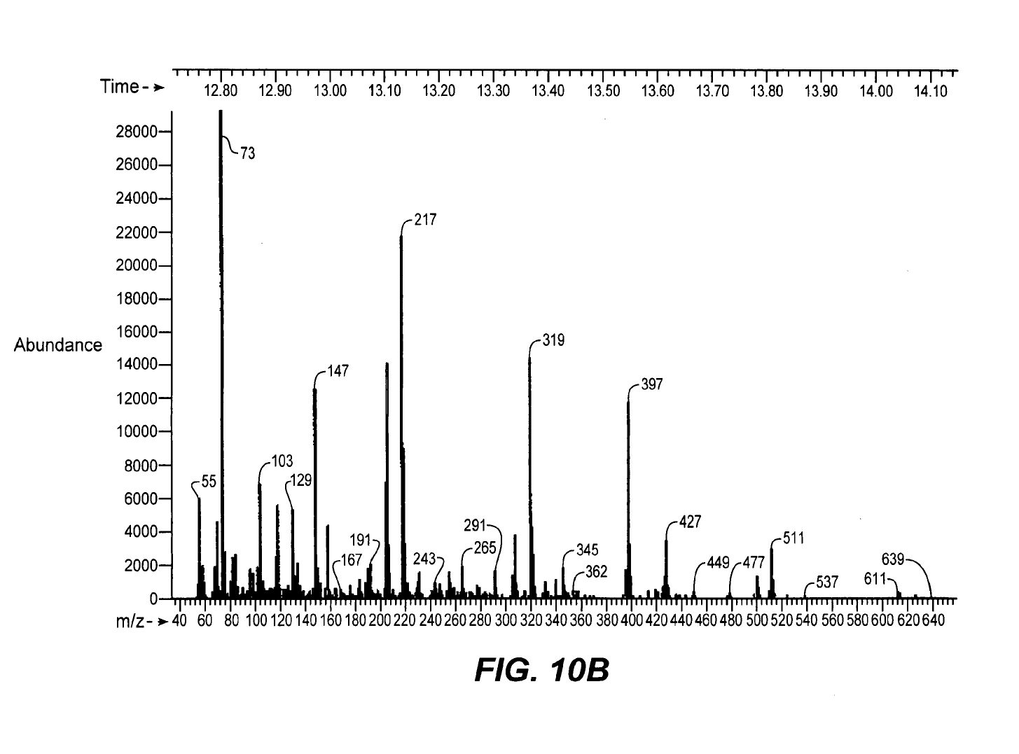

MS spectra of the peak marked sorbitol monooleate in FIG. 8 is shown in FIG. 10 and compared with the MS spectra of sorbitol monooleate.

HPTLC analysis of the reaction products indicate that a polar component has been formed during the incubation. GLC/MS analysis confirmed that sorbitol monooleate was formed. Sorbitol monooleate is a polar component with surface active properties that will act as a surfactant in water systems.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

Having described the some embodiments of the present invention, it will appear to those ordinarily skilled in the art that various modifications may be made to the disclosed embodiments, and that such modifications are intended to be within the scope of the present invention.

Those of skill in the art readily appreciate that the present invention is well adapted to carry out the objects and obtain the ends and advantages mentioned, as well as those inherent therein. The compositions and methods described herein are representative embodiments, are exemplary, and are not intended as limitations on the scope of the invention. It is readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by reference to some embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

What is claimed is:

1. A composition for producing a fragrant ester, comprising the following components:
a) an SGNH acyltransferase,
b) an alcohol substrate for said SGNH acyltransferase, and
c) an acyl donor;
wherein said SGNH acyltransferase catalyzes transfer of an acyl group from said acyl donor to said alcohol substrate to produce a fragrant ester in an aqueous environment.

2. The composition of claim 1, wherein said composition is an aqueous composition that further comprises said fragrant ester.

3. The composition of claim 1, wherein said alcohol substrate and said acyl donor and chosen to produce a particular fragrant ester.

4. The composition of claim 1, wherein said acyl donor is a C1 to C10 acyl donor.

5. The composition of claim 1, wherein said SGNH acyltransferase is immobilized on a solid support.

6. The composition of claim 1, wherein said composition is a dehydrated composition, and wherein said fragrant ester is produced upon rehydration of said composition.

7. The composition of claim 1, wherein said composition is a foodstuff.

8. The composition of claim 1, wherein said composition is a cleaning composition comprising at least one surfactant.

9. The composition of claim 1, wherein said composition is a cleaning composition comprising a hydrogen peroxide source.

10. A method of producing a fragrant ester, comprising combining:
a) an SGNH acyltransferase,
b) an alcohol substrate for said SGNH acyltransferase, and
c) an acyl donor;
wherein said SGNH acyltransferase catalyzes transfer of an acyl group from said acyl donor onto said alcohol substrate to produce said fragrant ester.

11. The method of claim 10, wherein said alcohol substrate and said acyl donor are selected to produce a particular fragrant ester.

12. The method of claim 10, wherein said acyl donor is a C2 to C10 acyl donor.

13. The method of claim 10, wherein said method comprises rehydrating said components after they are combined.

14. The method of claim 13, wherein said rehydration occurs during mastication.

15. The method of claim 10, wherein said SGNH acyltransferase, said alcohol substrate and said acyl donor are combined in an aqueous environment.

16. The method of claim 10, wherein said SGNH acyltransferase is immobilized on a solid support.

17. A method for the simultaneous generation of a bleaching agent and a fragrance comprising:
combining:
a) an SGNH acyltransferase,
b) an alcohol substrate for said SGNH acyltransferase, and
c) an acyl donor;
wherein said SGNH acyltransferase catalyzes transfer of an acyl group from said acyl donor onto said alcohol substrate to produce said fragrance and said bleaching agent.

18. The method of claim 17, wherein said bleaching agent is a peracid.

19. The method of claim 18, wherein said peracid is peracetic acid.

20. The method of claim 17, wherein said SGNH acyltransferase is M. smegmatis acyltransferase.

21. The method of claim 17, wherein said fragrance is an ester.

22. The method of claim 21, wherein said ester is a C2 to C6 ester of a primary alcohol.

23. The method of claim 17, wherein application of said bleaching agent to a stain results in removal of said stain.
